(19) United States
(12) Patent Application Publication
(10) Pub. No.: US 2023/0285739 A1
(43) Pub. Date: Sep. 14, 2023

(54) SYSTEMS AND METHODS FOR A PERISTALSIS HEART ASSIST PUMP

(71) Applicant: PHAP MEDICAL, LLC, Trabuco Canyon, CA (US)

(72) Inventors: Steve Smith, Trabuco Canyon, CA (US); Brian Babson, Long Beach, CA (US); Michael Saveliev, Huntington Beach, CA (US)

(21) Appl. No.: 18/182,282

(22) Filed: Mar. 10, 2023

Related U.S. Application Data
(60) Provisional application No. 63/319,170, filed on Mar. 11, 2022.

(51) Int. Cl. A61M 60/295; A61M 60/157

(52) U.S. Cl. CPC A61M 60/295; A61M 60/157

(57) ABSTRACT

Some embodiments of the disclosure are directed to a novel system for pumping liquid such as blood without damaging cells. In some embodiments, the system includes one or more inflatable elements such as balloons which force liquid from an implanted pump. In some embodiments, one or more elements are configured to directionally inflate. In some embodiments, the directional inflation enables pumping in a bidirectional manner. In some embodiments, the system includes a plurality of elements that inflate sequentially to pump liquid in one direction or another. In some embodiments, the one or more elements are coupled to a tube or stent.

### CROSS REFERENCE TO RELATED APPLICATION

This application claims the benefit and priority of U.S. Provisional Patent Application No. 63/319,170, filed Mar. 11, 2022, entitled “CARDIAC ASSIST PERISTALSIS PUMP SYSTEM,” which is incorporated herein by reference in its entirety.

### BACKGROUND

Various medical conditions can necessitate use of a cardiac assist device. One prior art device for pumping blood from the heart includes a small rotary impeller inside a tube which is inserted into the left ventricle. Impeller rotation results in the extraction of blood from the left ventricle into the aorta. However, because the impeller is rotating at a very high speed and is in direct contact with the blood, the impeller may damage blood cells.

Another type of blood pump includes a linearly reciprocating pump such as the one described in U.S. patent application Ser. No. 17/073,085. This pump works by expanding and contracting a diaphragm using an actuator rod. This creates an “umbrella” action where blood can flow around the collapsed diaphragm and then is pushed out as the diaphragm expands. While less impactful than an impeller, the linearly reciprocating pump lacks the ability to pump blood in more than one direction, and only a single diaphragm can be driven by the actuator rod.

Accordingly, there is a need in the art for a peristalsis heart assist pump that can be inserted into a body and minimize or eliminate damage to blood cells while also providing bidirectional flow.

### SUMMARY

In some embodiments, the disclosure is directed to a system for pumping blood that minimizes damage to blood cells. In some embodiments, the system comprises one or more element fluid pumps, one or more element tubes, one or more elements, and/or one or more stents. In some embodiments, the one or more elements are housed within the stent. In some embodiments, the one or more elements are configured to receive a fluid from the one or more element tubes. In some embodiments, the one or more element pumps are configured to execute an inflation and/or a deflation of the one or more elements via the one or more element tubes;

In some embodiments, the system further includes one or more element housings. In some embodiments, the one or more elements are housed in the one or more element housings. In some embodiments, the one or more element housings are housed within the stent. In some embodiments, the one or more element housings are housed within a tube and/or catheter. In some embodiments, the one or more elements are configured to pump a liquid though the one or more element housings as a result of the inflation and/or the deflation of the one or more elements. In some embodiments, the one or more elements are configured to execute a directional inflation. In some embodiments, the direction inflation is configured to force a liquid in one direction.

In some embodiments, each of the one or more elements comprise an element inlet end and an outlet end. In some embodiments, each the one or more elements are configured to enable an element inlet end to expand before an outlet end expands.

In some embodiments, at least one of the one or more elements include a tubular shape. In some embodiments, the tubular shape includes an element inlet end, an element outlet end, and an element hollow center portion. In some embodiments, the tubular shape is configured to enable a liquid to flow through the element hollow center portion. In some embodiments, the at least one element is configured to form the element hollow center portion when the at least one element is deflated.

In some embodiments, the system further includes a mandrel. In some embodiments, the mandrel is positioned within a center portion of the one or more element housings in the stent. In some embodiments, the one or more elements are coupled to the mandrel. In some embodiments, the one or more elements are configured to execute an inflation to cause one or more elements to expand from the mandrel toward the one or more element housings. In some embodiments, the expansion is configured to cause liquid to be pumped out of the housing in a single direction.

In some embodiments, a tube and/or a mandrel is configured to support both positive pressure and vacuum ranges in the same structure. In some embodiments, the mandrel includes one or more upstream pressure sensors 2016 and/or one or more downstream pressure sensors 2017. In some embodiments, the one or more pressure sensors 2016, 2017 are configured to sense liquid (e.g., blood) pressure. As used herein, references to a tube and/or mandrel are interchangeable when defining the metes and bounds of the system and also include a reference to internal structures such as fill tubes for element fluid. In some embodiments, a tube and/or a mandrel is configured to support gas volume consistent with desired cycle times by balancing inflation and deflation times. In some embodiments, a tube and/or a mandrel is configured to support a minimum of 500,000 cycles. In some embodiments, a tube and/or a mandrel includes a flexible material configured to be inserted to the left ventricle through the femoral artery. In some embodiments, a tube and/or a mandrel is configured to be abrasion resistant to minimum 500,000 cycles. In some embodiments, a tube and/or a mandrel is configured to be compatible with blood and other medical fluids added patients to blood. In some embodiments, a tube and/or a mandrel includes a Food and Drug Administration (FDA) compliant blood compatible material.

In some embodiments, one or more elements are shaped, formed, and/or configured to directionally inflate. In some embodiments, one or more elements include a variable material thickness. In some embodiments, one or more elements include variable material durometers. In some embodiments, one or more elements include a material compatible with blood and/or medical fluids (e.g., fluids added to a patient's blood). In some embodiments, one or more elements include an FDA compliant material. In some embodiments, one or more elements include Pellethane and/or similar materials. In some embodiments, one or more elements are abrasion resistant to a minimum of 600,000 cycles. In some embodiments, one or more elements are configured to inflate and deflate a minimum of 600,000 cycles.

In some embodiments, the one or more elements include a first element and a second element. In some embodiments, the first element comprises a first inflated volume. In some embodiments, the second element comprises a second inflated volume. In some embodiments, the first inflated volume is less than the second inflated volume. In some embodiments, the first element is positioned before the second element in the one or more housings relative to pumping direction. In some embodiments, inflation of the first element is configured to close a housing inlet end into the one or more housings. In some embodiments, inflation of the second element is configured to pump liquid from a first outlet end of the first element to a second outlet end of the second element.

In some embodiments, the system further comprises a third element. In some embodiments, the third element comprises a third inflated volume. In some embodiments, the third inflated volume is less than the second inflated volume. In some embodiments, the third element is positioned after the second element in the one or more housings relative to pumping direction. In some embodiments, inflation of the third element is configured to close a housing outlet end out the one or more housings.

In some embodiments, the system further comprises one or more of a controller, a first element, and a second element. In some embodiments, the one or more elements include the first element, and the second element. In some embodiments, the controller is configured to execute a first inflation of the first element before executing a second inflation of the second element. In some embodiments, the system includes a third element. In some embodiments, the one or more elements include the third element. In some embodiments, the controller is configured to execute a third inflation of the third element after executing the second inflation of the second element.

In some embodiments, the system includes a graphical user interface (GUI). In some embodiments, the GUI is configured to enable a user to execute a first pumping sequence configured to pump liquid in a first direction. In some embodiments, the GUI is configured to enable a user to execute a second pumping sequence configured to pump liquid in a second direction. In some embodiments, the controller is configured to execute a first deflation of the first element after executing a second deflation of the second element. In some embodiments, the controller is configured to execute a third deflation of the third element before executing a second deflation of the second element. In some embodiments, the controller is configured to execute an overlapping inflation of one or more elements. In some embodiments, the controller is configured to execute an overlapping deflation of one or more elements.

### DRAWINGS DESCRIPTION

FIG. 1 illustrates a system overview according to some embodiments.

FIG. 2 shows a non-limiting example graphical user interface 101 according to some embodiments.

FIG. 3 shows an element filling operation 1A-5B executed by the system in a tubular element arrangement 300 according to some embodiments.

FIG. 4 depicts a liquid pump including a stent arrangement comprising one or more elements and one or more stents according to some embodiments.

FIG. 5 illustrates the operation of the stent arrangement which is similar to the operation of the balloon within the tube arrangement previously described according to some embodiments.

FIG. 6 shows a 1st filling step 1F and a 1st deflating step 1D in a balloon cycle according to some embodiments.

FIG. 7 shows a 2nd filling step 2F and a 2nd deflating step 2D according to some embodiments.

FIG. 8 shows a 3rd filling step 3F and a 3rd deflating step 3D according to some embodiments.

FIG. 9 is a continuation of the inflation and deflation illustrations of FIG. 8 according to some embodiments.

FIG. 10 is a continuation of the inflation and deflation illustrations of FIG. 9 according to some embodiments.

FIG. 11 shows a final inflation step 6F and a final deflation step 6D, which is also the beginning of the cycle as shown in FIGS. 5 and 6 according to some embodiments.

FIGS. 12 and 13 show a non-limiting rendering of a single element configuration according to some embodiments.

FIG. 14 shows a plural balloon system comprising two elements according to some embodiments.

FIG. 15 shows a three-balloon arrangement according to some embodiments.

FIG. 16 shows a sequential element arrangement comprising a plurality of elements 16nth according to some embodiments.

FIG. 17 shows a tapered shaped element according to some embodiments.

FIG. 18, in some embodiments, as this pocket collapses during inflation the angle of the cone increases as more of the inflated balloon fills the inner portion of a tube, stent, and/or lumen.

FIG. 19 shows one or more check elements according to some embodiments.

FIG. 20 shows a liquid pump which includes a mandrel according to some embodiments.

FIG. 21 shows a plurality element fluid pump arrangement according to some embodiments.

FIG. 22 depicts steps implemented by one or more configurations described herein according to some embodiments.

FIG. 23 illustrates a computer system enabling or comprising the systems and methods in accordance with some embodiments.

FIG. 24 shows a pleated element arrangement in a partially inflated configuration according to some embodiments.

FIG. 25 illustrates the directional inflation of a pleated element according to some embodiments.

FIG. 26 shows a non-limiting example inlet check element in an inflated configuration according to some embodiments.

FIG. 27 shows a non-limiting example side and font view for a pumping element according to some embodiments.

FIG. 28 shows a non-limiting example inflated outlet check element according to some embodiments.

### DETAILED DESCRIPTION

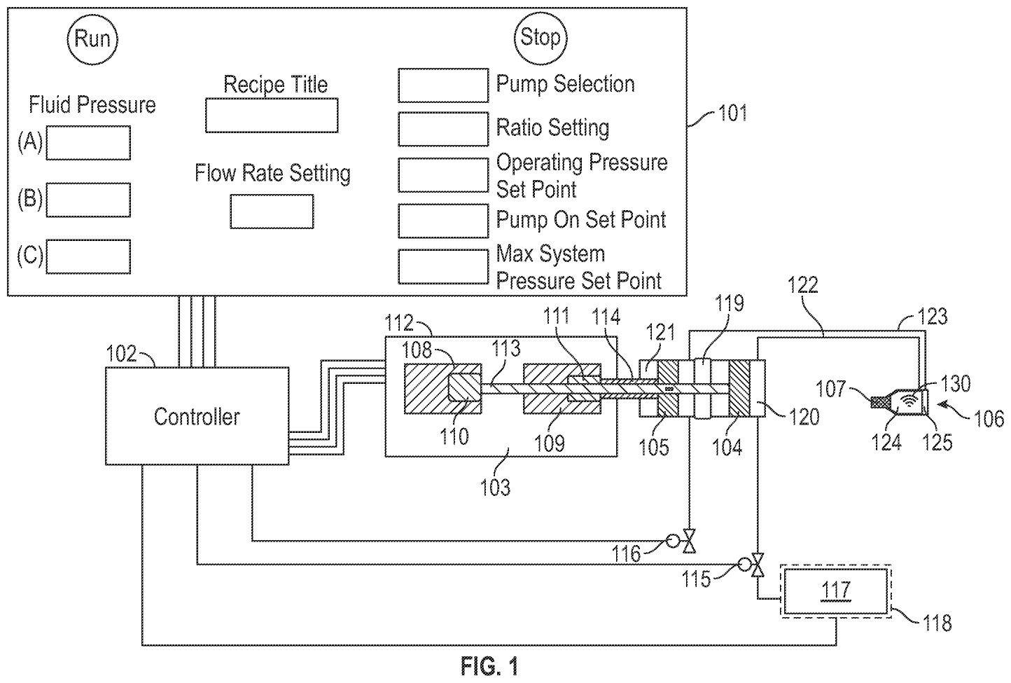

As shown in FIG. 1, in some embodiments, the peristalsis heart assist pump (PHAP) system (hereafter the “system”) includes one or more: graphical user interfaces 101 (GUIs), controllers 102, element fluid pumps 103, pistons 104, 105, liquid pumps 106, and/or stints 107. As used herein, the term “element fluid” refers to the medium used to inflate and/or deflate an element, which may be a liquid or a gas according to some embodiments. The term “liquid” refers to the medium that is being pumped by the liquid pump 106, which includes blood as a non-limiting example according to some embodiments. In some embodiments, the liquid pump 107 is configured to be used as a peristalsis heart assist pump. In some embodiments, the liquid pump 107 comprises the one or more elements 124, 125.

In some embodiments, the one or more elements 124, 125 include one or more balloons 124, 125. As used herein, any reference to a balloon is also a reference to the broader genus element, where the terms are interchangeable for the purposes of defining the metes and bounds of the system. As used herein, a balloon includes any elastic material that is configured to expand when a fluid pressure is supplied and contract when the fluid pressure is removed. In some embodiments, the elastic material includes medical grade material suitable for insertion into the human body.

In some embodiments, one or more elements 124, 125 are configured to control the flow of liquid. In some embodiments, the one or more elements 124, 125 are inflatable and/or collapsible under pressure and/or vacuum, respectively. In some embodiments, non-limiting example elements include inflatable elements, collapsible elements, check elements, umbrella elements, flexible elements, and/or magnetic elements, where the modifier (e.g., inflatable) serves to describe the element's function and/or structure.

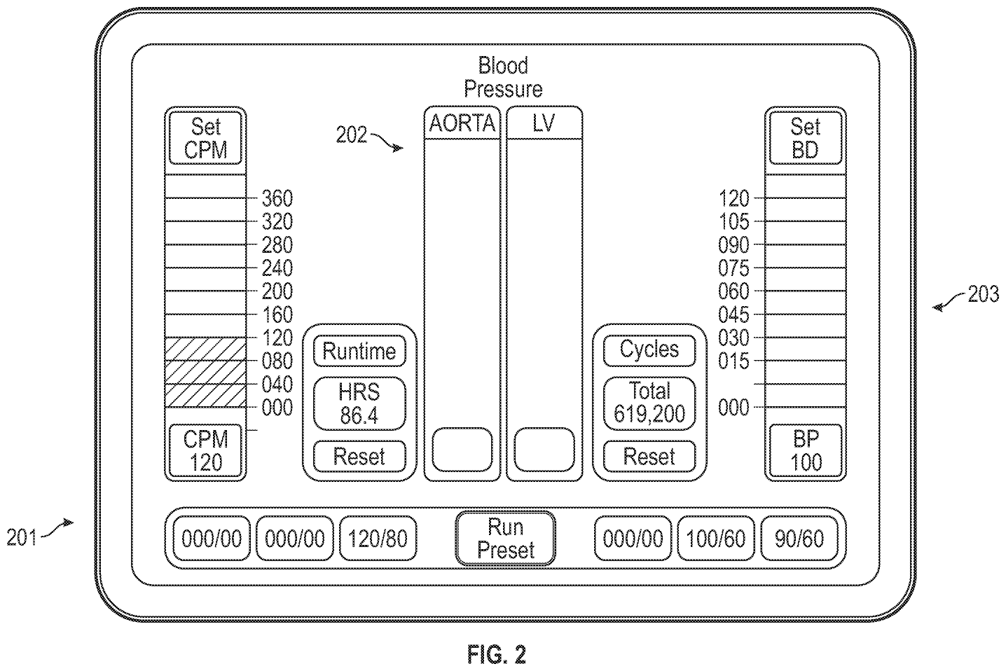

The system includes a graphical user interface 101 (GUI) configured to accept one or more user inputs and/or display one or more system settings according to some embodiments. FIG. 2 shows a non-limiting example graphical user interface 101 according to some embodiments. In some embodiments, the graphical user interface 101 is electronically (e.g., wired, wireless) to a controller 102 which is configured to actuate one or more system components including one or more element fluid pumps 103.

In some embodiments, the one or more element fluid pumps 103 comprise one or more linear motors. In some embodiments, the one or more linear motors each include one or more coils 108, 109 and/or one or more cores 110, 111. In the non-limiting example shown in FIG. 1, the system includes a novel dual coil linear motor 103 that comprises a first coil 108 and a second coil 109 arranged along the same axis within a motor housing 112. In some embodiments, positioned within the first coil 108 and second coil 109 is a first core 110 and second core 111, respectively. In some embodiments, the first coil 108 is configured to move the first core 110 along a hollow axis of the first coil 108 through the generation of a first magnetic field. In some embodiments, the second coil 109 is configured to move the second core 111 along a hollow axis of the second coil 109 through the generation of a second magnetic field.

In some embodiments, the dual coil linear motor 103 is configured to actuate more than one piston 104, 105 (substantially) simultaneously. In some embodiments, a first drive shaft 113 with a first outer diameter is connected to the first core 110 and a first piston 104. In some embodiments, a second drive shaft 114 with a second drive shaft inner diameter is connected to a second core 111 and a second piston 105. In some embodiments, both the first piston 104 and second piston 105 are housed in the same element fluid pump 103.

In some embodiments, the second drive shaft inner diameter is defined by a second drive shaft hollow portion where the second drive shaft hollow portion extends axially along the second drive shaft 114. In some embodiments, the second core 111 includes a second core inner diameter defined by a second core hollow portion where the second core hollow portion extends axially along the second coil. In some embodiments, the second piston 105 includes a second piston inner diameter defined by a second piston hollow portion where the second piston hollow portion extends axially along the second piston 105.

In some embodiments, the second drive shaft inner diameter, the second coil inner diameter, and/or the second piston inner diameter are greater than the first outer diameter, such that the dual coil linear motor 103 is configured to enable the first drive shaft 113 to pass through the second coil 109, the second drive shaft 114, and/or the second piston 105 to connect to first piston 104 as shown in FIG. 1.

In some embodiments, the controller 102 is configured to control one or more fluid (e.g., gas) supply valves 115, 116 which are each configured to supply fluid to the liquid pump 106 from one or more fluid supplies 117. In some embodiments, the one or more fluid supplies 117 include one or more heater jackets 118 configured to regulate temperature via the controller 102. In some embodiments, such as the non-limiting example in FIG. 1, the controller 102 is configured to control a first fluid supply valve 115 and a second fluid supply valve 116. In some embodiments, the element fluid pump 103 includes one or more piston chamber housings 119 one or more pistons chambers 120, 121. In some embodiments, the element fluid pump 103 includes a first piston chamber 120 housing the first piston 104 and a second piston chamber 121 housing the second piston 105.

In some embodiments, the non-limiting example graphical user interface 101 is configured to display one or more system parameters including one or more fluid setpoints 201 and/or one or more sensor measurements 202. In some embodiments, the graphical user interface 101 is configured to display one or more element pressure setpoints 203 for one or more piston chambers 120, 121 and/or one or more elements 124, 125. In some embodiments, the graphical user interface 101 is configured to enable a user to enter one or more gas setpoints by selecting one or more icons shown in FIG. 2. In some embodiments, the graphical user interface 101 is configured to enable a user to enter one or more fluid setpoints for the first piston chamber 120 and/or the second piston chamber 121. In some embodiments, a gas setpoint includes one or more of a pressure, a flowrate, a temperature, and/or an actuation status. In some embodiments, the graphical user interface is configured to display and/or control one or more of electrocardiogram (ECG), heart rate, inlet blood pressure, outlet blood pressure, flow rate, cycle settings, active cycle rate, gas pressure settings each element. active gas pressure each element, error codes, alarm, pump identification (ID), date, time, run time, operator ID, patient ID and/or asset location.

In some embodiments, a reciprocating action of a piston 104, 105 is configured to increase pressure and/or force fluid one or more elements 124, 125 by decreasing the volume of at least a portion of a piston chamber. In some embodiments, the reciprocating action of a piston 104, 105 is configured to decrease pressure and/or retrieve gas from the one or more elements by increasing the volume of at least a portion of a piston chamber. In some embodiments, the controller 102 is configured to maintain a substantially constant mass of fluid within a piston chamber 104, 105. In some embodiments, the GUI 101 is configured to enable a user to enter a pressure range and/or display one or more warnings/and or alerts on the GUI 101 if the range is exceeded. In some embodiments, the controller 102 is configured to initiate emergency pressure control instructions configured to remove any fluid from a first fill tube 122 and/or second fill tube 123 and/or stop the operation of a piston 104, 105.

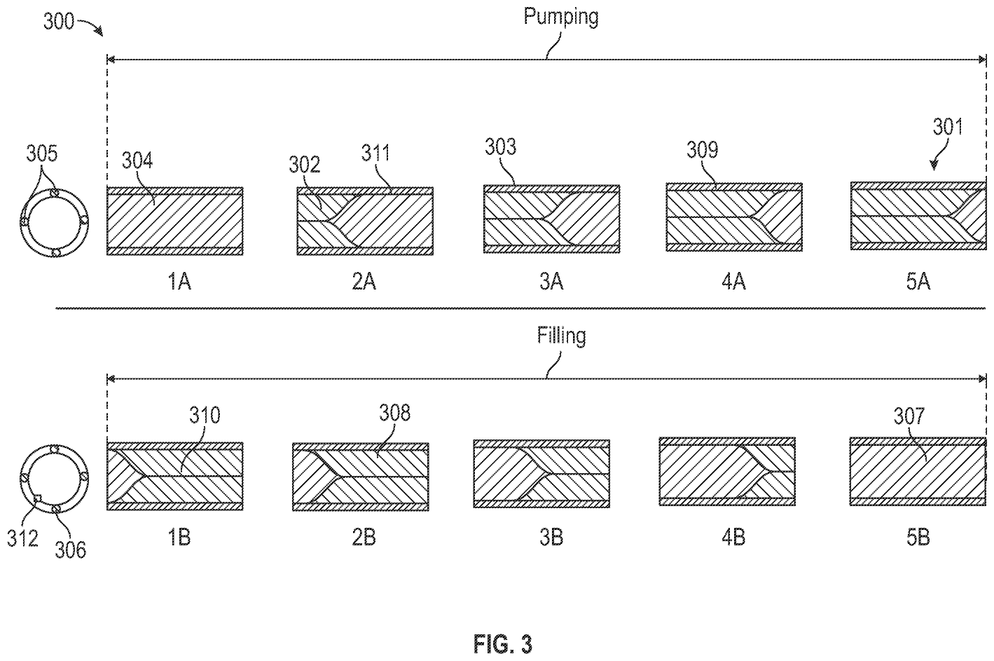

FIG. 3 shows an element filling operation 1A-5B executed by the system in a tubular element arrangement 300 according to some embodiments. In some embodiments, as shown in the non-limiting example in FIG. 3, the system includes a single element 301. In some embodiments, the element includes a single balloon 302 positioned within at least a portion of a tube 303. In some embodiments, the length of the tube 303 extends past the length of the single balloon 303. In some embodiments, a stent 107 surrounds the balloon as shown in FIG. 1. In some embodiments, the tube 303 is configured to transport a liquid 304 such as blood, as a non-limiting example. In some embodiments, one or more fill tubes 305 supplying fluid from one or more element fluid pumps 103 connect to the single balloon 302 by extending through a fill tube 305 to the balloon 302 to actuate a filling and pumping operation of the balloon 302. In some embodiments, a plurality of fill tubes 305 supplied to a single balloon 302 provides the benefit of applying pressure to a balloon 302 from a plurality of directions.

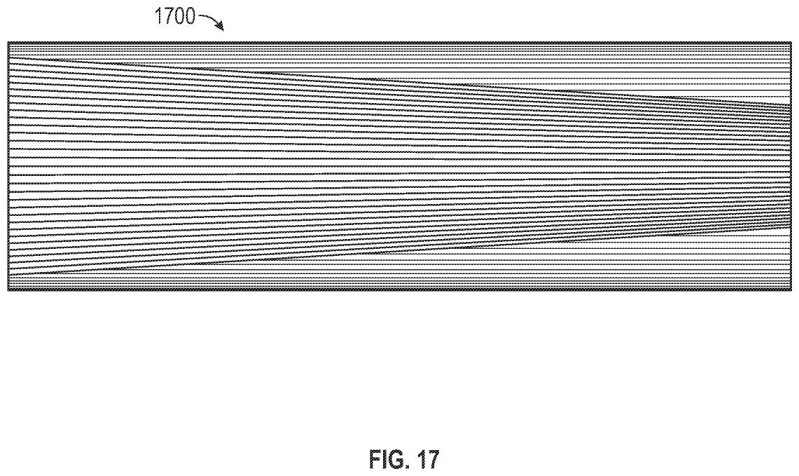

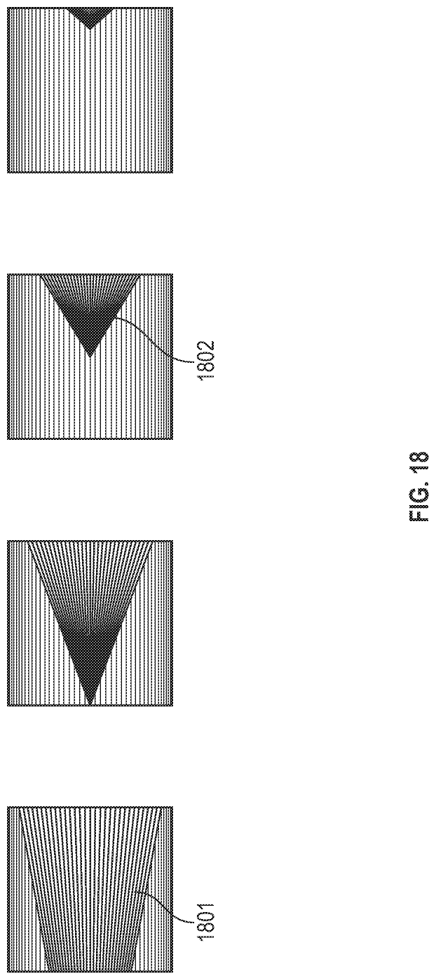

In some embodiments, each of the one or more fill tubes 305 comprise one or more fill orifices 306 configured to deliver the element fluid to one or more balloons 302. In some embodiments, a fluid conduit with one or more fill tubes and one or more fill orifices are referred to herein as lumen. Although FIG. 3 depicts a single balloon 302, as further described herein the system comprises a plurality of balloons (elements) in series according to some embodiments. Any property of an element described according to some embodiments is understood to be applicable to any other element arrangement (e.g., plurality arrangement) according to some embodiments. In some embodiments, each of the one or more fill tubes 305 are configured to supply fluid to a separate balloon in a plurality arrangement as described later. In some embodiments, one or more fill tubes 305 are configured to supply fluid to a first balloon of a plurality of balloons, and one or more other fill tubes are configured to supply fluid to one or more other balloons of the plurality of balloons. In some embodiments, the system is configured to at least partially trap and/or transport a fluid in a bi-directional manner by actuating one or more balloons in the series, with non-limiting examples of a bi-directional arrangement are illustrated in FIGS. 17 and 18.

Still referring to FIG. 3, in some embodiments, one or more balloons 302 comprise a balloon hollow core 307 which give the balloon a tubular shape when at least partially deflated at a first pump stage 1A, which is the same as a final pump stage 5B. In some embodiments, one or more balloons 302 are configured to directionally inflate. In some embodiments, the one or more balloons each comprise one or more different densities and/or thicknesses in at least a portion a balloon wall along the balloon's length. FIG. 17 illustrates an exaggerated density profile according to some embodiments. In some embodiments, at least a portion of a balloon's outer wall 308 is attached to the fluid conduit 309. In some embodiments, directional inflation and/or directional deflation cause a pumping action of fluid within at least a portion of the fluid conduit 309.

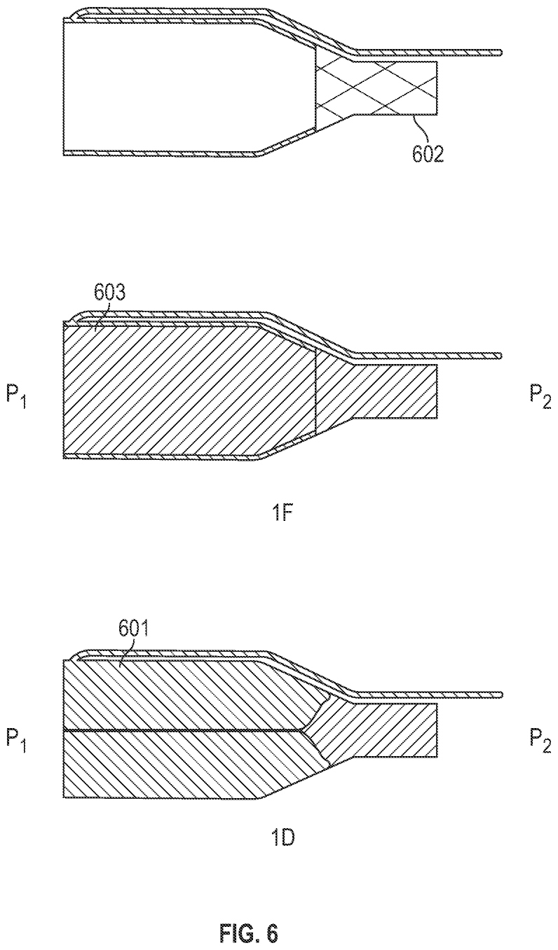

In some embodiments, upon an actuation of a piston 104, the balloon is configured to at least partially seal a first end of the fluid conduit 309 (which may also include and/or be an element housing in some embodiments) by pressing the balloon inner faces toward each other at a stage 2A. FIGS. 6-12 provide an enlarged view of this action according to some embodiments, noting that features from any of the shown figures in all portions of this disclosure are interchangeable and readily incorporable with each other. Stages 3A-5A show continued expansion of the balloon 302 pushing the fluid to the right according to some embodiments. In some embodiments, this increases the pressure of the moving fluid greater than the pressure being supplied by incoming fluid from the left. In some embodiments, as a piston return stroke starts to remove pressure from the balloon 302 the incoming fluid from the left pushes balloon inner faces 310 back toward the fluid conduit wall 311 as the piston pulls the gas from one or more fill orifices 306. In some embodiments, one or more balloons, one or more fill tubes, and/or one or more fluid conduits comprise one or more sensors 312 (e.g., Wheatstone bridge) configured to feedback one or more balloon parameters to the controller in the form of electrical signals, electromagnetic signals, light signals, and/or fluid pressure signals. In some embodiments, a balloon parameter includes one or more of a balloon pressure, a balloon tension, a balloon activation, a percent inflation, and/or any other parameter that can be interpreted from a signal from a sensor.

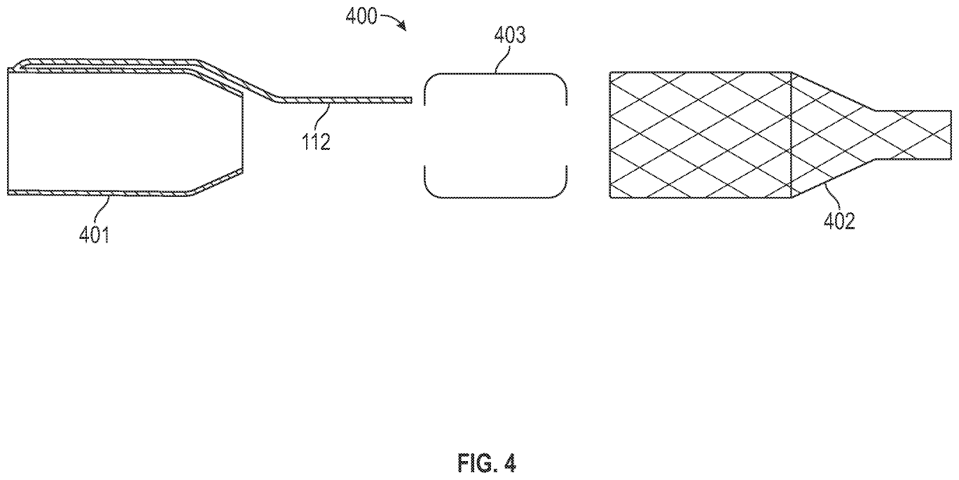

As shown in FIG. 4, in some embodiments, the liquid pump 106 includes a stent arrangement 400 comprising one or more elements 401 and one or more stents 402 (e.g., nitinol stents as a non-limiting example). In some embodiments, size is a limiting factor for the liquid pump 106. In some embodiments, the stent arrangement 400 is configured and/or sized to be inserted into a human body. In some embodiments, the stent arrangement 400 (e.g., balloon) is configured and/or sized to be inserted into a human body. In some embodiments, one or more check elements (e.g., balloons, magnetic valves) are minimized in size to enable a larger main element which can induce a larger flow as further described below.

In some embodiments, system comprises one or more elements 124, 125 within the stent 107. In some embodiments, the system comprises an element housing 403 (309), where the one or more elements 401 are located within the element housing 403. In some embodiments, the element housing 403 is configured to be placed within the stent 402. In some embodiments, the element housing 403 is flexible and/or collapsible. In some embodiments, the element housing 403 and/or the one or more elements 401 are configured to be removed from the stent after the stent is placed inside a patient. In some embodiments, the element housing is coupled to a mandrel. In some embodiments, the element housing is coupled to the stent 402.

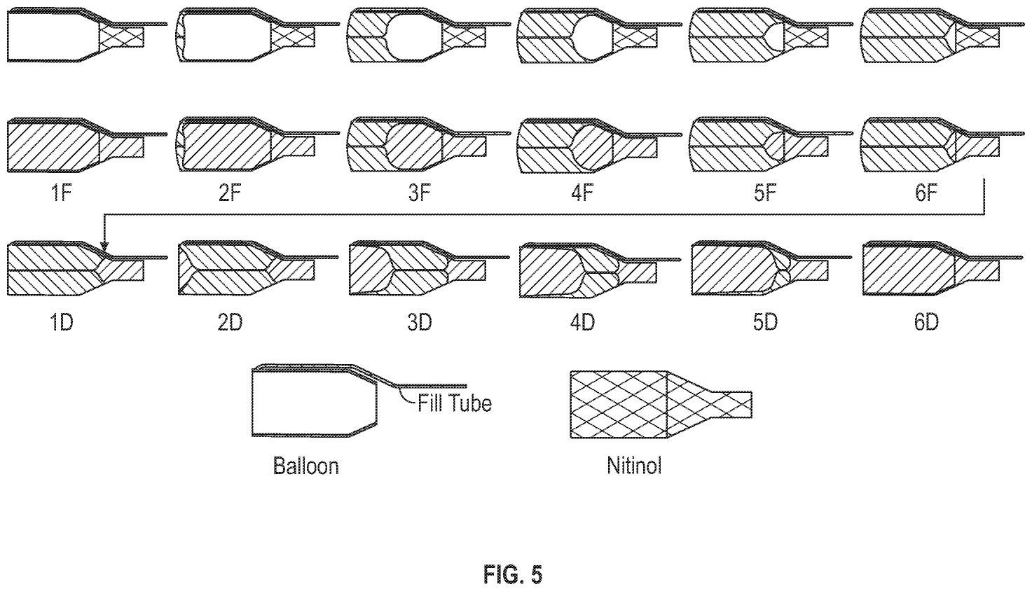

In some embodiments, a stent 402 (107) comprises a temperature dependent memory metal configured to expand when exposed to body temperature for a pre-determined time. FIG. 5 illustrates the operation of the stent arrangement 400 which is similar to the operation of the balloon within the tube arrangement 300 previously described according to some embodiments. Enlarged details of the stent arrangement 400 according to some embodiments can be seen in FIGS. 6-15.

While not to be limited to any principle or application of physics, a discussion of operation of a stent (and/or tube) arrangement for a single element and/or portion of a plurality of elements is described below. Inflation and deflation steps are illustrated together to highlight the behavior of the balloon structure under opposite conditions.

In some embodiments, systems and methods described herein are directed to a number of cycles (e.g., per min) needed to achieve a desired flow rate while delivering a desired output pressure given a fixed pumping volume per cycle. In some embodiments, factors other than the fixed volume of the pumping element that influence cycle time include the fluid pressure required to inflate each element and the time to deflate each element. In some embodiments, the higher the final inflation pressure the longer the deflation time given the use of dual function lumens. In some embodiments, one area that effects the pressure requirement is loss in the lumen due to the size of the lumens. In some embodiments, empirical data has revealed that a minimum of 40% of the overall element fluid lumens area allocated to the pumping element, a minimum of 10% to the inlet check element, and a minimum of 10% of the overall gas lumens to the outlet check element is sufficient to achieve the desired pumping effect. Although the function of a check element is to prevent the flow of liquid, the inflation action of a check element, which may include a directional inflation, also provides pumping action according to some embodiments.

FIG. 6 shows a 1^{st} filling step 1F and a 1^{st} deflating step 1D in a balloon cycle according to some embodiments. In some embodiments, these two steps represent when an element 601 is at a completely deflated and fully inflated configuration, respectively. In some embodiments, when completely deflated at step 1F, the pressure on either side of the stent 602 P₁ and P₂ are equal, but generally lower than desired in the implant environment. In some embodiments, after the liquid 603 has been forced completely from the element 601 and/or stent 602, P₁ is slightly higher than P₂ at step 1D as pressure builds at the opening.

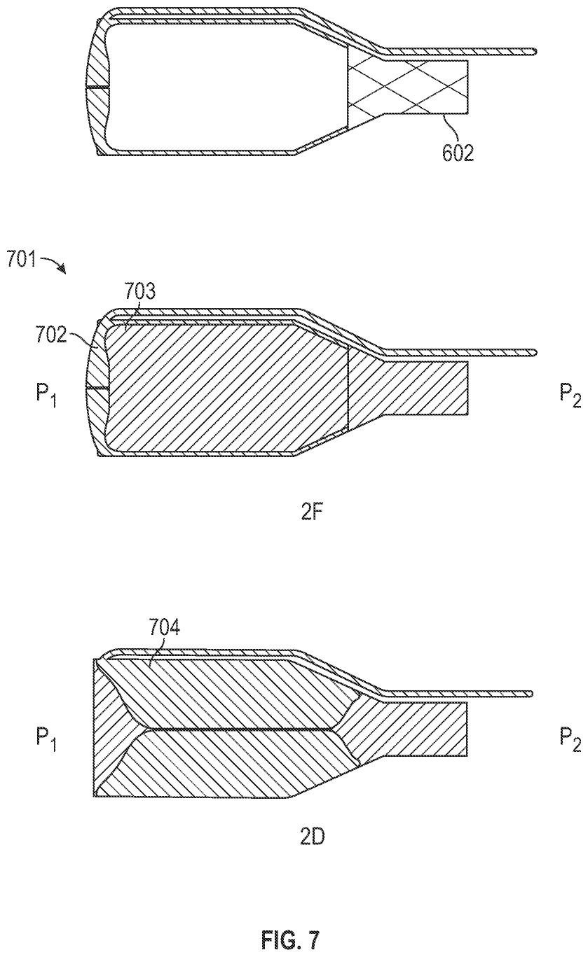

FIG. 7 shows a 2^{nd} filling step 2F and a 2^{nd} deflating step 2D according to some embodiments. In some embodiments, at a 2^{nd} filling step the upstream opening 701 is closed by the element gate portion 702 trapping the liquid 703 in the balloon's central portion. In some embodiments, at a 2^{nd} deflating step the balloon gate portion 702 is deflated before the balloon body portion 704. In some embodiments, the deflation of the gate portion 702 first is at least partially aided by one or more of the element's structure (e.g., density) and the higher pressure at P₁.

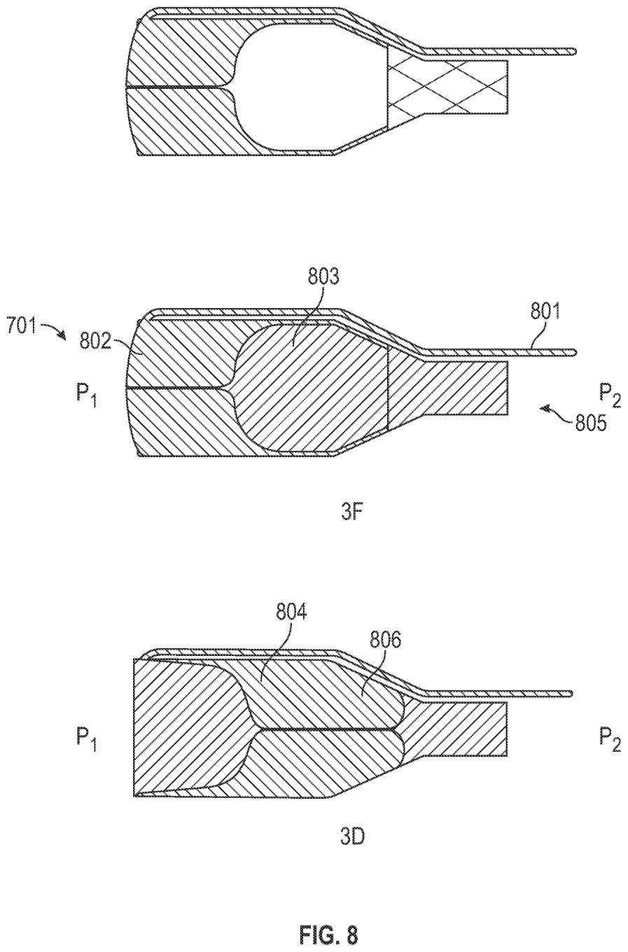

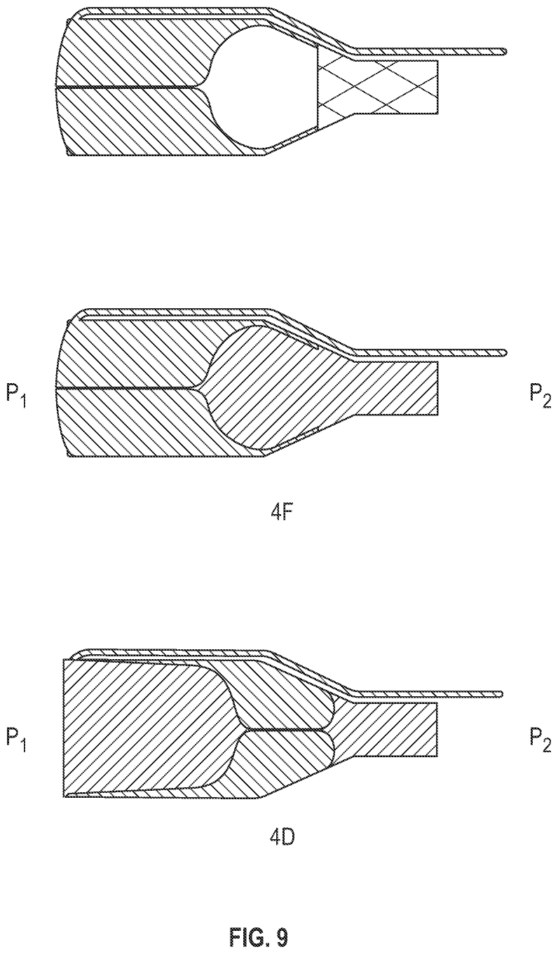

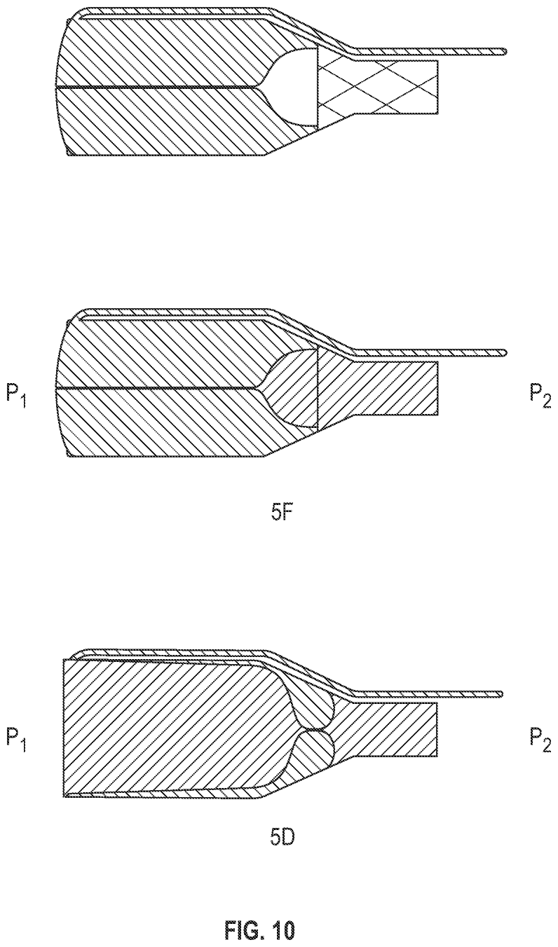

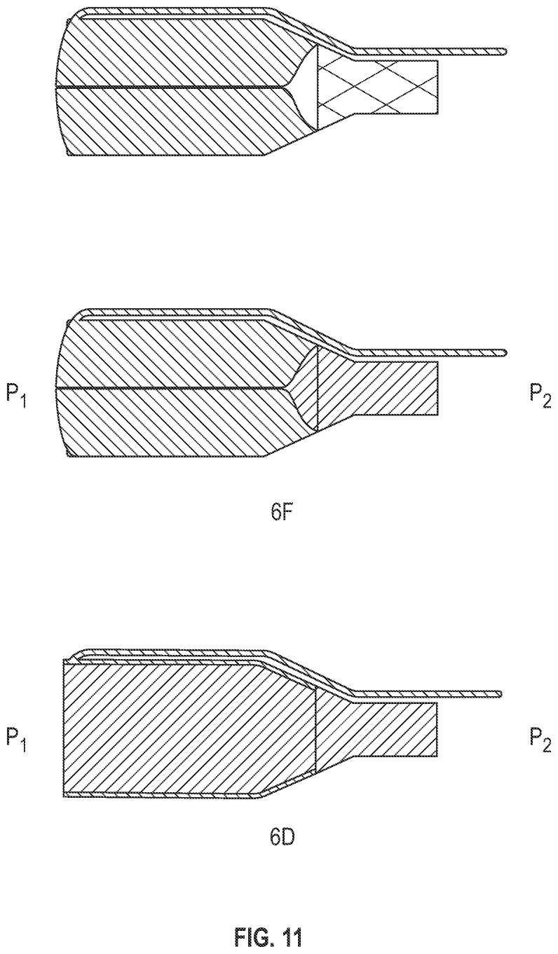

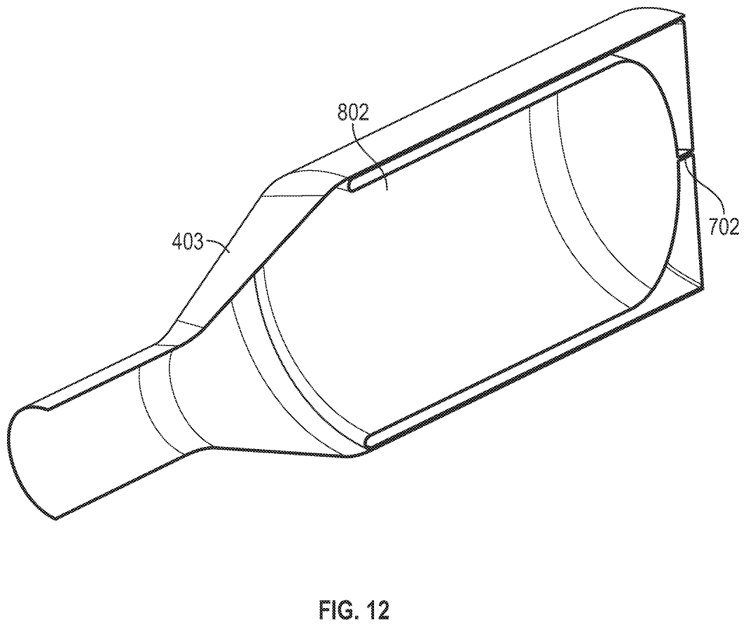

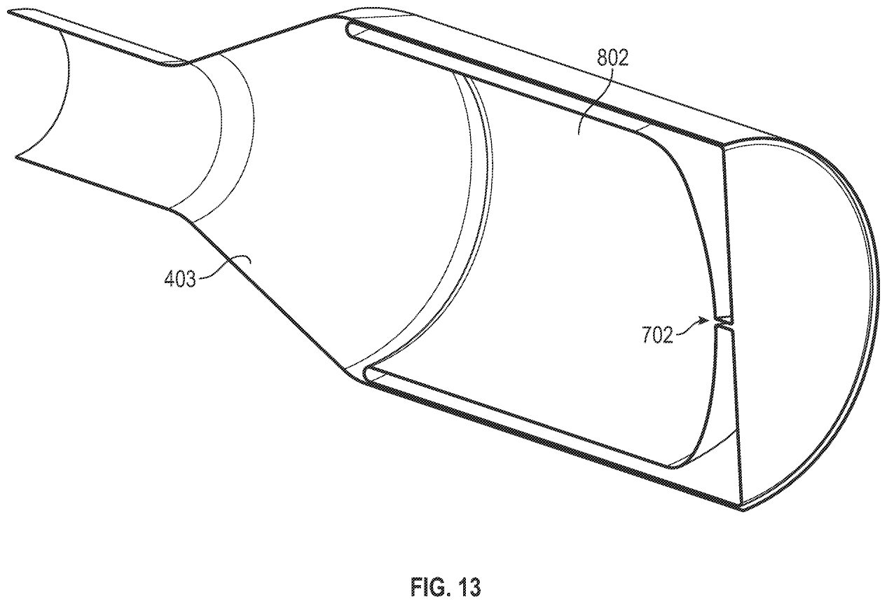

FIG. 8 shows a 3^{rd} filling step 3F and a 3^{rd} deflating step 3D according to some embodiments. In some embodiments, the pressure of fluid supplied from the fill tube 801 and/or the structure of the element at filling step 3F causes the element body portion 804 to inflate toward the downstream opening 805. In some embodiments, this inflation direction forces the liquid 803 out of the downstream opening increasing the overall pressure downstream. In some embodiments, at a 3^{rd} deflation step 3D, the upstream pressure P₁ pushes the liquid 803 against the balloon body portion 804 which pushes element fluid 806 toward the downstream opening end maintaining a closed balloon configuration at the downstream opening 805. FIG. 9 is a continuation of the inflation and deflation illustrations of FIG. 8. FIG. 10 is a continuation of the inflation and deflation illustrations of FIG. 9. FIG. 11 shows a final inflation step 6F and a final deflation step 6D, which is also the beginning of the cycle as shown in FIGS. 5 and 6 according to some embodiments. FIGS. 12 and 13 show a non-limiting rendering of a single element configuration according to some embodiments. Although the inflation direction is shown coming from the wider end of the element, in some embodiments the inflation direction is the opposite direction. In some embodiments, the element is configured to use the narrower section of the element housing and/or stent to force the element end to close first, aiding the directional inflation.

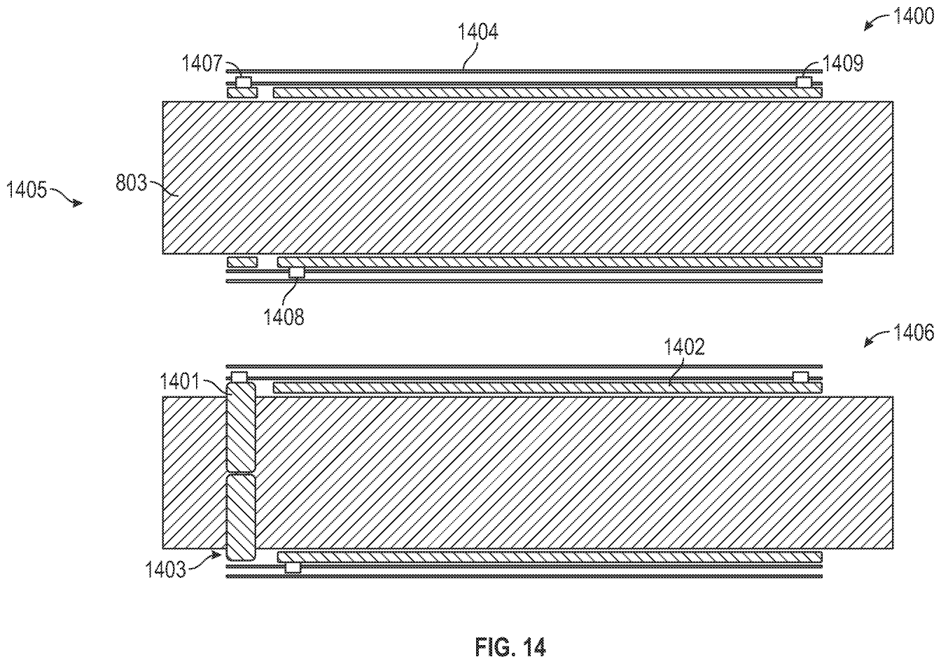

FIG. 14 shows a plural balloon system 1400 comprising two elements 1401, 1402 in this non-limiting example: note that the same directional inflation operation in this tube arrangement applies to the stent arrangement shown in FIG. 1, which illustrates the interchangeability of the different arrangements described herein. In some embodiments, the controller 102 is configured to inflate the first element 1401 before inflating the second element 1402. In some embodiments, the controller 102 is configured to inflate the second element 1402 when the first element 1401 at least partially seals a first end 1403 of the tube 1404, thereby preventing further fluid flow from upstream 1405 (e.g., in the direction of natural blood flow) in this non-limiting example. In some embodiments, the controller 102 is configured to inflate the second balloon in a directional manner previously described with respect to FIGS. 3 and 5. In some embodiments, the natural downstream flow 1406 aids in the formation of the balloon as previously described.

In some embodiments, the one or more balloons includes a single orifice 1407. In some embodiments, the one or more balloons include two or more orifices 1408. In some embodiments, a tube 1404 with multiple orifices each supplying a specific amount of fluid is referred to as a lumen. In some embodiments, the second element 1402 comprises an upstream orifice 1408 and a downstream orifice 1409. In some embodiments, controller is configured to apply positive pressure to the upstream orifice 1408 while applying a negative pressure to the downstream orifice 1409. In some embodiments, a negative pressure is configured to hold the internal walls of the second element 1402 against one another while the positive pressure fills the upstream portion of the second element 1402.

In some embodiments, cycle time is influenced by the dwell time in between each element's actuation action driven by inflation time and deflation time before the next element can activate. In some embodiments, a 1 to 100 millisecond overlap between actuation of individual elements has been determined to ensure smooth flow with little to no back flow while decreasing cycle time. In some embodiments, the system is configured to begin inflating a pump element 1402 before the inflation of a check element 1401 is complete. In some embodiments, the system is configured to begin the inflation of a check element 1401 before a pump element 1402 has been fully inflated. In some embodiments, the system is configured to begin deflating a pump element 1402 before the deflating of a check element 1401 is complete. In some embodiments, the system is configured to begin the deflating of a check element 1401 before a pump element 1402 has been fully deflated.

In some embodiments, the system is configured to optimize a sequence of inflation and deflation for each element to produce a cycle time for the device that supports a target flow rate with given a targeted device size. In some embodiments, the system is configured to vary and/or supply different element fluid pressures to each element to be able to allow each element to perform the inflation and deflation in a way that reduces cycle time.

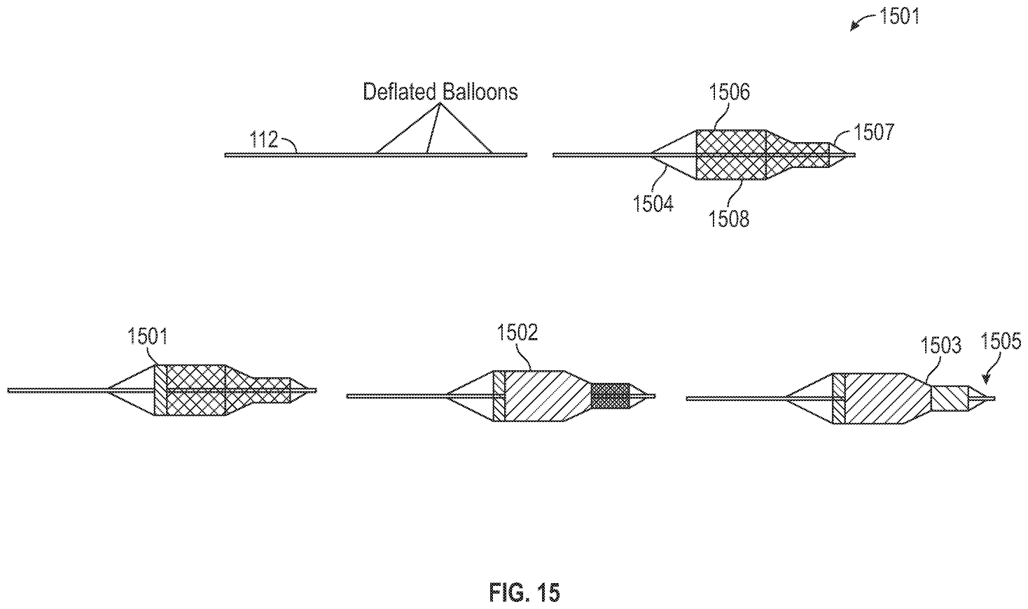

FIG. 15 shows a three-balloon arrangement 1500 according to some embodiments. In a non-limiting example where element 1501 is the inlet check element and element 1503 is the outlet check element, both must be synchronized with pumping element 1502. In some embodiments, pumping element 1502 comprises an inflated volume at least twice that of check element 1501 and/or 1502. In some embodiments, in each case the inlet check element 1501 and outlet check element 1502 must execute their function to allow the pumping element 1502 to perform in a sequential way which generally results in reduced fluid movement time in the cycle. In some embodiments, the system is configured to supply different pressure settings to each element. In some embodiments, the system is configured to supply higher pressure to the check elements to create a quicker inflation time. In some embodiments, due to different inflated volumes of each element 1501, 1502, and/or 1503, the opportunity for overlapping activation exists by utilizing the pressurization time of the pumping element against the shorter inflation and deflation time of the check/pump elements. The check elements also provide some pumping action as a result of reducing volume according to some embodiments.

In some embodiments, the system is configured to apply a vacuum to an element. In some embodiments, the vacuum assist deflation which influences cycle time which can be used to control cycle time of each element. In some embodiments, the system is configured to generate a maximum vacuum (i.e., highest the system will allow) to shorten the cycle time.

In some embodiments, the fluid (e.g., gas) lumens have a dual function of delivering fluid at a set pressure and removing fluid under vacuum. In some embodiments, the size of the fluid passage influences the cycle time. In some embodiments, each element size affects available volume to pump fluid (e.g., blood) and by minimizing the size of the check/pump elements the volume pumped per cycle increases. In some embodiments, empirical testing has shown an inlet check element at a maximum of 15% inflated volume of the pumping element inflated volume, and an outlet check valve of a maximum of 10% of the pumping element inflated volume produces acceptable results.

In some embodiments, when inserted to a specified location in a human body approximately the controller is configured to apply 20-30 mmHg of positive pressure exist on the large end of the balloon housing. In some embodiments, the controller is configured to inflate each element in a sequence that causes a decrease in volume and displacing the fluid through the small end 1507 of the element housing and/or stent 1508. In some embodiments, the controller is configured to deflate the elements in a sequence, drawing fluid into the element housing filling at least a portion or the entirety of each void left by a balloon's deflation.

In some embodiments, the three-balloon arrangement 1501 comprises a central pumping balloon 1502 that is has an expanded volume greater than an upstream check element 1501 and/or a downstream check element 1502. In some embodiments, a pumping sequence starts with the controller inflating all elements. In some embodiments, the controller 102 is configured to deflate the pumping balloon 1502 to create a vacuum. In some embodiments, the controller 102 is configured to deflate the upstream check balloon 1501 (e.g., in approximately 1/10 second or less) to create an additional vacuum. In some embodiments, the (combined) vacuum draws fluid (e.g., blood under 20-30 mmHG of positive pressure) into the balloon housing 1506. In some embodiments, the controller 102 is configured to deflate the downstream check element 1503 (e.g., in approximately 1/10 second or less and/or while the fluid is in motion) adding additional momentum from the vacuum created to eject the fluid from the balloon housing. In some embodiments, the momentum this arrangement provides creates a “slingshot effect” that results in greater fluid volume output than the sum of the balloon volumes and/or balloon housing volume. In some embodiments, the controller 102 is configured to repeat these initiation instructions one or more times to create a pumping cycle. In some embodiments, the repeating action creates a generally or completely sinusoidal wave fluid flow and/or fluid pressure profile. In some embodiments, the controller 102 is configured to initiate the inflating and deflating sequence in an opposite manner such that the downstream check element 1503 deflates before the upstream check element 1501 deflates thereby enabling flow in a bi-directional manner. In some embodiments, a controller initiated pump sequence can include program instructions for a right-to-left and/or left-to-right inflation/deflation sequence where each balloon is inflated and/or deflated in a sequential order. In some embodiments, the GUI is configured to enable a user to program the controller to inflate and/or deflate a balloon in any sequence described herein.

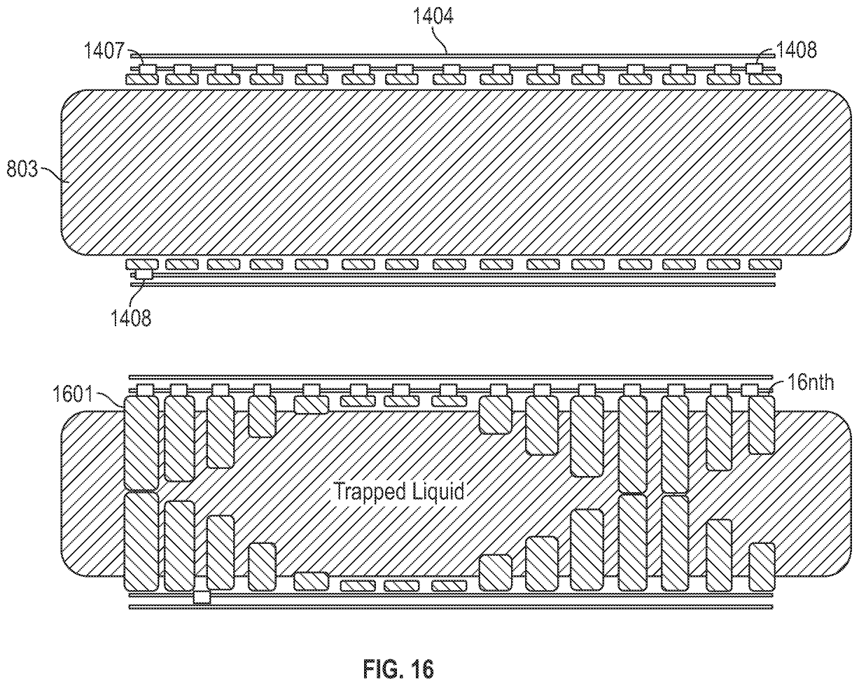

FIG. 16 shows a sequential element arrangement 1600 comprising a plurality of elements 16nth according to some embodiments. In some embodiments, each element 16nth is approximately the same size. In some embodiments, one or more elements are different sizes and/or displace different volumes. In some embodiments, each element can exhibit the same or very different inflation characteristics depending on wall thicknesses, durometers, and other typical balloon characteristics. In some embodiments, the controller 102 is configured to acuate one or more elements, which include a first element 1601 up to an nth element 16nth (i.e., any number of elements) in a peristalsis sequence where trapped fluid is moved along a path by supplying fluid (e.g., gas) in a predetermined order. Although in this example the fluid is trapped between two or more fully inflated elements, in some embodiments one or more of the plurality of elements are configured to directionally inflate in a similar shape and flow profile as previously described with respect to a single, dual, and/or three element arrangements previously described and illustrated in at least FIG. 5.

In some embodiments, the controller 102 is configured to generate 20-30 mmHg of vacuum at the distal (larger diameter) end of the peristalsis heart assist pump. In some embodiments, the controller 102 is configured to initiate a pumping cycle similar to those previously described herein according to some embodiments such as with the three element arrangement. In some embodiments, the controller 102 is configured to implement a sequence to generate fluid momentum by inflating and deflating the elements in an order as previously described.

In some embodiments, an element shape when deflated and/or inflated influences pumping action in a pumping element and/or fluid element. In some embodiments, a pumping element shape allow for the inflation to occur in a desirable direction which results in the fluid flow moving from inlet to outlet in a more controlled way.

In some embodiments, one or more elements described herein include a tapered shape and/or is configured to inflate in a tapered shape. FIG. 17 shows a tapered shaped element 1700 according to some embodiments. In some embodiments, the tapered shape is configured to create a cone shaped volume pocket 1801 during inflation. FIG. 18 illustrates the cone shaped profile 1801 according to some embodiments. As shown in FIG. 18, in some embodiments, as this pocket 1801 collapses during inflation the angle 1802 of the cone increases as more of the inflated balloon fills the inner portion of a tube, stent, and/or lumen. In some embodiments, the tapered shape of the tapered element is configured to increase the velocity of the trapped fluid as the trapped fluid is moved from the inlet to the outlet end as the pocket 1801 collapses. In some embodiments, the increased velocity results in an increase in fluid pressure. In some embodiments, a tapered element 1700 which causes inflation to be directed to one direction is desirable in achieving a controlled flow and reduces high velocity areas in the fluid flow. In some embodiments, empirical data has shown taper angle range of 1 degree up to 60 degrees at any point along 80% of the element length will achieve a desirable result. In some embodiments, one or more check elements and/or pump elements described here include a tapered element 1700. In some embodiments, a tapered element with a pleated design is described below.

In some embodiments, the check/pump element shapes are configured to inflate in a substantially perpendicular direction with sufficient surface area force to at least partially seal the tube (e.g., lumen) with minimum gas volume. In some embodiments, element area ribbing is configured to direct inflation while limiting expansion to enable increased pumping element capacity. In some embodiments, check element inflation is configured to provide pumping action. In some embodiments, the pumping action is bidirectional.

In some embodiments, element material thickness, durometer value, and/or ribbing surface are configured to provide benefit in controlling inflation and/or configured to provide additional benefit of encouraging the delated element against a mandrel to decrease deflated volume. In some embodiments, a tapered thickness of material for the pumping element provides similar benefits for controlling flow direction by having the thinner material area inflate first resulting in the controlled directional inflation (see FIGS. 17 and 18). In some embodiments, material thickness differential of minimum 5% to 300% in a taper or intermittent fashion along the element has been empirically determined to provide a desired effect.

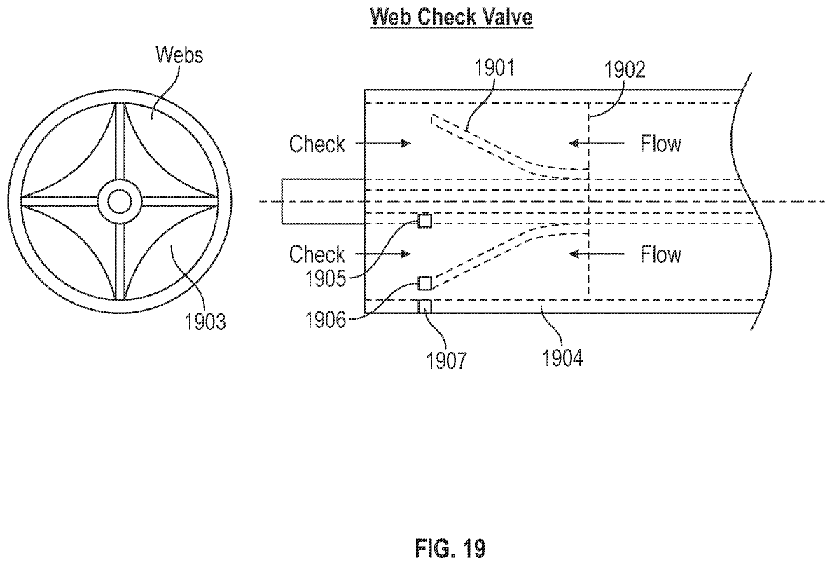

FIG. 19 shows one or more check elements 1901 according to some embodiments. In some embodiments, one or more check elements 1901 are not balloons. In some embodiments, the one or more check element 1901 are web check elements 1901. In some embodiments, the one or more check elements 1901 are configured to curve upward and/or block liquid flow in one direction. In some embodiments, the one or more check elements 1901 are configured to enable flow in one direction. In some embodiments, the one or more check elements are configured to be pushed down by one or more inflating balloons, such as a pump element in a single balloon arrangement. In some embodiments, one or more check elements are a combination of balloons and web elements. In some embodiments, the one or more web check elements 1901 are configured to cause at least a portion of the one or more balloons 1902 to lift and/or interfere with a direction of flow while allowing flow in the other direction when deflated. In some embodiments, the one or more web check elements 1901 are the same material as the balloon 1902. In some embodiments, the one or more check elements 1901 are a different material as the balloon 1902. In some embodiments, the one or more web check elements 1901 include a different density as the surrounding material. In some embodiments, the liquid pump 106 comprises one or more balloons 1902 and/or one or more web check elements (e.g., 1-2 elements) 1901. In some embodiments, the one or more check elements 1901 include one or more conventional medical fluid check elements configured for insertion into a patient. In some embodiments, the one or more check elements 1901 are configured to flatten during positioning into a patient and/or expand to a functional configuration once positioned in a patient.

In some embodiments, the one or more web check elements 1901 include one or more expandable portions 1903 configured to be inflated by an element fluid. In some embodiments, the one or more expandable elements 1903 are housed in a stent 107. In some embodiments, the one or more expandable elements 1903 are configured to act as a check valve. In some embodiments, the one or more expandable elements 1903 are configured to act as a check valve when deflated. In some embodiments, the stent 107 comprises a nitinol stint. In some embodiments, one or more stents 107 are housed inside a catheter 1904, which may also be a lumen and/or tube according to some embodiments. In some embodiments, the one or more stents 107, check elements 1901, 1501 element housings 403, and/or elements 16nth are configured to project from the catheter 1904 once in position. In some embodiments, system is configured to enable the one or more stents 107, check elements 1501, element housings 403, and/or elements 124, 125 to be collapsed, rehoused, and/or be crimped back inside the catheter 1904 during extraction from the patient.

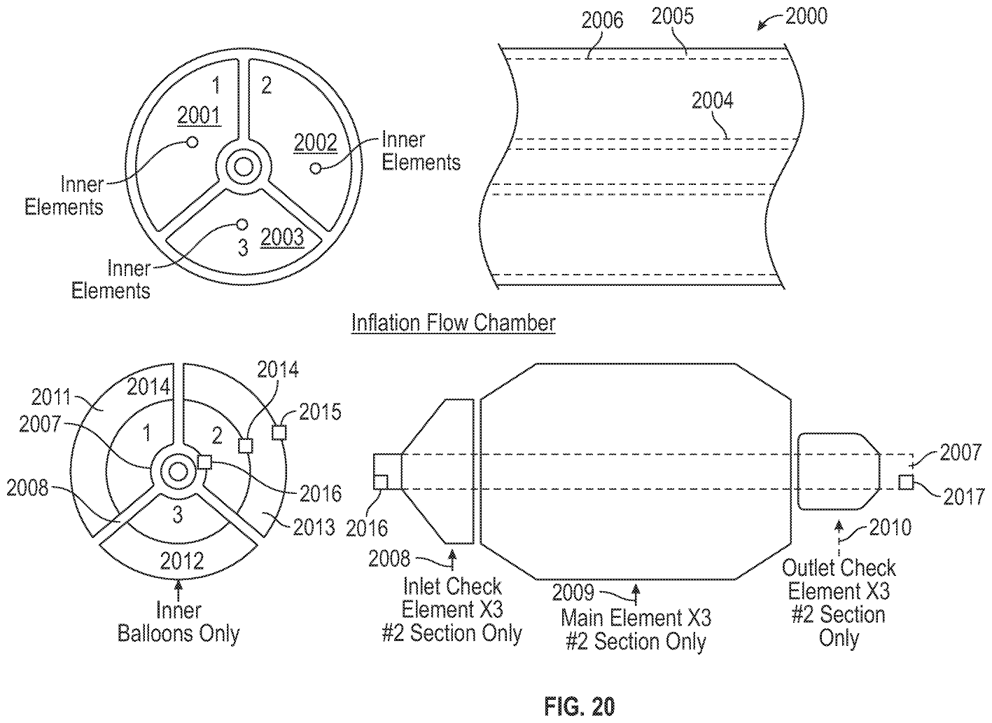

FIG. 20 shows a liquid pump 2000 in the form of a mandrel flow chamber 2000 according to some embodiments. In some embodiments, the flow chamber 2000 includes one or more inflatable elements 2001, 2002, 2003 according to some embodiments. In some embodiments, the flow chamber 2000 includes one or more fill lines 2004 (i.e., lumens) incorporated into the flow chamber walls 2006 and/or the mandrel 2007. In some embodiments, element fluid is delivered from the mandrel to the flow chamber walls 206 via one or more arms 2008. In some embodiments, the one or more check elements 2008, 2010 and/or one or more pump elements 2009 are configured to be attached and/or are coupled to the inside of the flow chamber wall 2006 and/or the mandrel 2007. In some embodiments, the one or more check elements 2008, 2010 and/or one or more pump elements 2009 are configured to inflate toward the center of the flow chamber 2011. In some embodiments, the one or more check elements and/or one or more pump elements are configured to inflate downward and/or away from the flow chamber wall 2006.

In some embodiments, the flow chamber 2011 comprises a mandrel 2007. In some embodiments, the one or more check elements 2008, 2010 and/or one or more pumping elements 2009 are configured to be attached and/or are coupled to the mandrel 2007. In some embodiments, the mandrel 2007 includes one or more arms 2008 extending the inner diameter of the flow chamber 2011. In some embodiments, the one or more arms 2008 are each configured to form a separate chamber 2012, 2013, 2014 in the flow chamber 2011. In some embodiments, the one or more check elements 2008, 2010 and/or one or more pumping elements 2001, 2002, 2003 are configured to inflate upward and/or outward from the mandrel 2007 to the flow chamber wall 2006. FIG. 20 shows both arrangements where 1, 2, and 3 are either the elements inflating outward or the void as the elements inflate toward the mandrel.

In some embodiments, the catheter 1904 is configured to couple to the liquid pump 106. In some embodiments, the liquid pump 106 includes a plurality of fill tubes 305 configured to feed element fluid (e.g., gas, liquid) to the one or more check elements 1501, 1503 and/or one or more pumping elements 1502. In some embodiments, one or more fill tubes 305 are configured to function as a guide for wire and/or an access for wire. In some embodiments, the one or more check elements 1501, 1503 and/or one or more pumping elements 1502 are configured to be inflated using one or more pressurized gas cylinders, valves, and/or compressed gas sources.

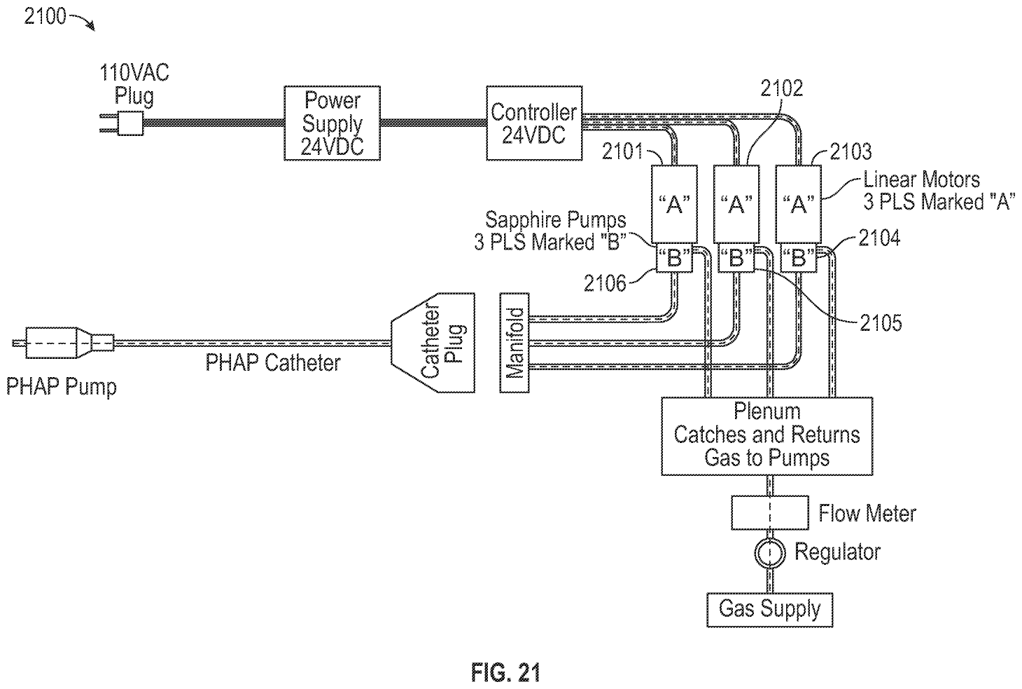

FIG. 21 shows a plurality element fluid pump arrangement 2100 according to some embodiments. In some embodiments, the system includes a plurality of element fluid pumps 2101, 2102, 2103 are configured to inflate and/or deflate the one or more check elements 1501, 1503 and/or one or more pump elements 1502 as previously described. In some embodiments, one or more pump pistons 2104, 2105, 2106 comprise sapphire and/or a similar material manufactured to a tolerance that needs no valve seals to enable the element fluid pumps 2101, 2102, 2103 to create pressure on a forward cycle and/or a vacuum on the reverse cycle. In some embodiments, the plurality element fluid pump arrangement 2100 and/or the dual linear pump 103 arrangement comprise a closed loop system configured to recirculate and/or reuse element fluid in a cyclical manner.

In some embodiments, the system comprises one or more linear motors, rotary motors, linear motion devices, and/or rotary motion devices configured to power one or more element fluid pumps 2101, 2102, 2103 to inflate and/or deflate various aspects of the system according to some embodiments as previously described. In some embodiments, one or more element fluid pumps 2101, 2102, 2103 are configured to generate a vacuum to increase the speed of deflation.

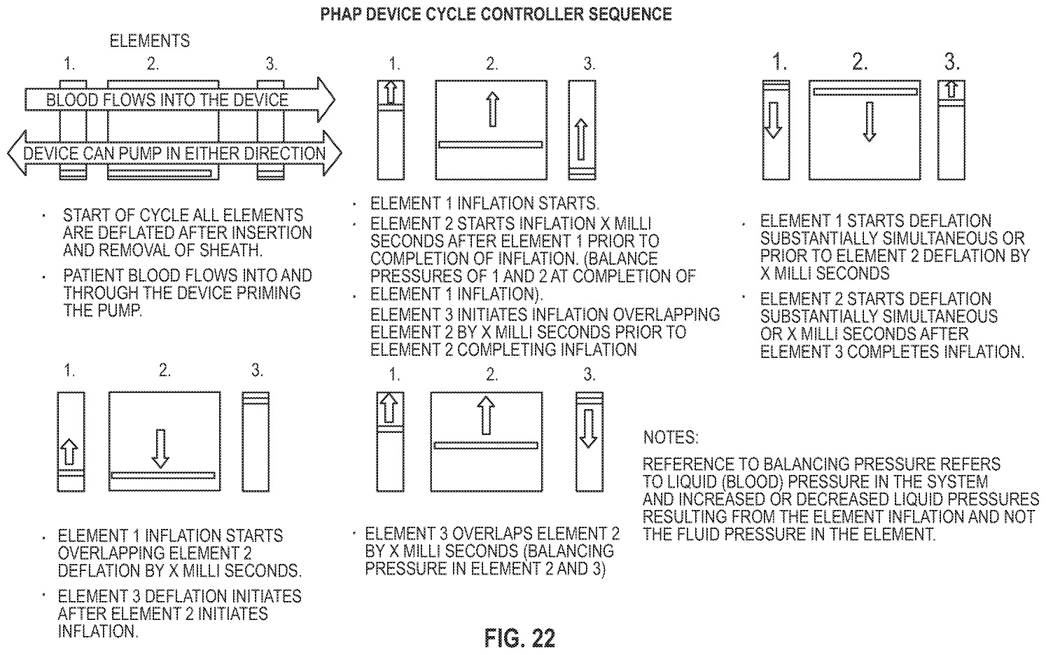

In some embodiments, the system comprises one or more computers comprising one or more processors and one or more non-transitory computer readable media. In some embodiments, the one or more non-transitory computer readable media include instructions stored thereon that cause the one or more computers to implement one or more programming steps by the one or more processors. FIG. 22 depicts steps implemented by one or more configurations described herein according to some embodiments.

In some embodiments, a step includes executing an inflation sequence. In some embodiments, the inflation sequence includes a sequence for inflating two or more elements. In some embodiments, a step includes executing a deflation sequence. In some embodiments, the deflation sequence includes a sequence for deflating two or more elements. In some embodiments, the inflation sequence for an element is different than a deflation sequence for a elements. In some embodiments, a step includes executing an independent inflation and/or deflation command to a single elements. In some embodiments, a step includes executing multiple single elements commands to different elements in a pre-determined pattern.

In some embodiments, a step includes executing an automatic and/or a manual mode. In some embodiments, a manual mode includes instructions to generate a graphical user interface (GUI) configured to enable an operator to manually set and/or change one or more system settings (e.g., desired resulting pressure). In some embodiments, one or more system settings includes element fluid and or liquid pump speed and/or blood pressure set points. In some embodiments, an automatic mode includes instructions executed by the one or more processors to maintain a blood pressure value. In some embodiments, the automatic mode includes instructions to automatically change its cycle rate (e.g., increase/decrease) to maintain a blood pressure value as conditions within a patient change.

In some embodiments, the liquid pump 107 includes one or more sensors 312. In some embodiments, the one or more sensors 312 are located in the one or more of the proximal and distal ends of the liquid pump 107. In some embodiments, the one or more sensors 312 are configured to monitor the blood pressure in the lower ventricle (LV) and/or aorta. In some embodiments, the instructions cause the computer to receive input from the one or more sensors to implement one or more controls (e.g., when in automatic mode).

In some embodiments, the system includes a display (e.g., color touch screen) configured to display the GUI 101. In some embodiments, the GUI 101 comprises one or more control functions for the system. In some embodiments, the GUI 101 comprises a blood pressure reading in PSI and/or mmHg.

In some embodiments, the liquid pump 107 includes a communication device 130. In some embodiments, the communication device 130 is configured to send one or more electronic transmissions to one or more controllers 102. In some embodiments, the communication device 130 is configured to send one or more electronic transmissions from the implant site and/or an area proximate the one or more components inside a patient. In some embodiments, an electronic transmission comprises a wireless signal. In some embodiments, the controller 102 comprises a receiver configured to receive the (wireless) electronic transmission. In some embodiments, an electronic transmission comprises data from one or more sensors 312. In some embodiments, an electronic transmission comprises a pump identification. In some embodiments, the pump identification is configured to verify the authenticity of the peristalsis heart assist pump.

In some embodiments, one or more components (e.g., any and/or all) described herein include a communication device 130. In some embodiments, the communication device 130 includes a pump identification device. In some embodiments, a pump identification device is configured to send patient data including one or more patient identification and/or medical details about the patient, the peristalsis heart assist pump, medical history, and/or any conventional information stored within the communication device 130. In some embodiments, the communication device 130 is configured to receive data through an electronic transmission. In some embodiments, the communication device 130 is configured to update, store, and/or replace data stored on one or more communication device non-transitory computer readable media with the received data. In some embodiments, the communication device 130 comprises one or more of a radio frequency identification (RFID) device, a Bluetooth® low energy (BLE) device, a near field communication device (NFC), and and/or an ultra-wide band (UWB) device, as a non-limiting examples.

In some embodiments, the liquid pump 107 includes one or more check valves. In some embodiments, the one or more check valves are used in place of one or more balloons and or inlet and/or outlet check elements 1901. In some embodiments, one or more check valves include a collapsing valve configured to collapse against a wall and/or mandrel to allow and/or stop flow. In some embodiments, a collapsing valve design includes one or more of a modified umbrella, (multi-segmented) duck bill (e.g., FIG. 19), and other flexible material valve design.

In some embodiments, the peristalsis heart assist pump comprises one or more electromagnetic valves. In some embodiments, the electromagnetic valves are configured to control inlet and/or outlet flow through the peristalsis heart assist pump. In some embodiments, the electromagnetic valves are configured to operate in conjunction with the one or more check elements 1901. In some embodiments, the liquid pump 107 is configured to pump fluid using the element fluid force and/or use electromagnetic elements 1905, 1906, 1907 to provide inlet and outlet check valve functions. In some embodiments, the liquid pump 107 comprises one or more magnets 1905, 1906, 1907 configured to attract to one or more other magnets and/or ferromagnetic materials positioned in the area of the liquid pump 107 inlet and/or outlet. In some embodiments, the mandrel 2007 and/or wall 2006 comprises one or more magnets 1905, 1907 configured to attract and/or repel one or more magnets 1906 and/or ferromagnetic materials 1906 coupled to one or more check valve elements 1901. In some embodiments, the one or more magnets 1905, 1907 include electromagnets configured to generate a magnetic field upon receiving an applied electrical current. In some embodiments, the electrical current is supplied and/or controlled by the one or more controllers 102. In some embodiments, an electromagnet 1905, 1907 is configured to repel the one or more magnets 1906 on the check element 1901 to open the check element 1901 (e.g., during a reverse flow). In some embodiments, an electromagnet 1905, 1907 is configured to attract the one or more magnets 1906 on the check element 1901 to open the check element 1901.

In some embodiments, the pumping element 2001, 2002, 2003 comprises one or more pump magnets 2014 and/or ferromagnetic materials 2014 configured to control pumping action through liquid pump 107. In some embodiments, the mandrel 2007 comprises one or more mandrel electromagnets 2016 configured to attract one or more magnets 2014 and/or ferromagnetic material 2014 integral and/or coupled to one or more pumping elements 2001, 2002, 2003. In some embodiments, the one or more mandrel magnets 2016 and/or one or more wall magnets 2015 are configured to enable the one or more pumping elements 2001, 2002, 2003 to directionally expand in the direction of the magnetic attraction. In some embodiments, the controller is configured to initiate one or more magnetic actuation sequences similar to the pneumatic sequences described herein to create directional fluid flow. In some embodiments, the one or more controllers 102 are configured to initiate one or more magnetic attraction and/or repulsion sequences in conjunction with one or more fluid (e.g., pneumatic) actuation sequences.

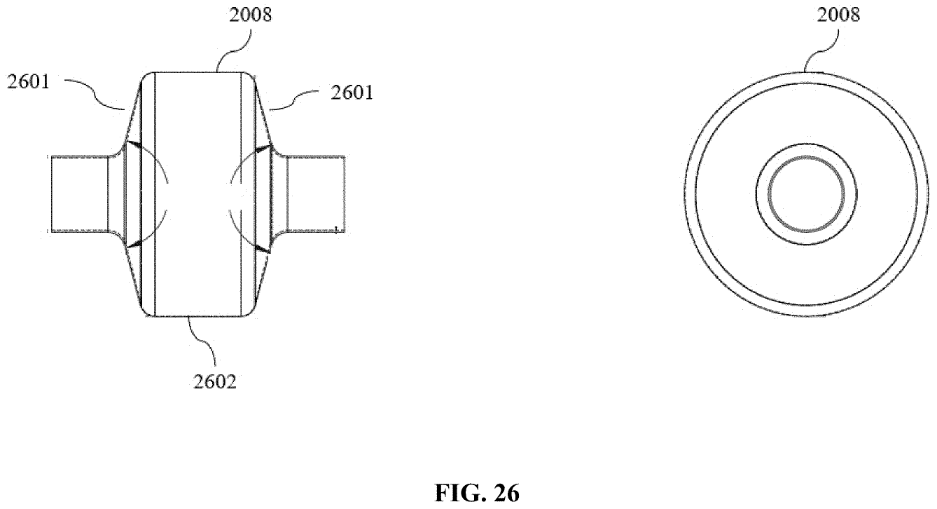

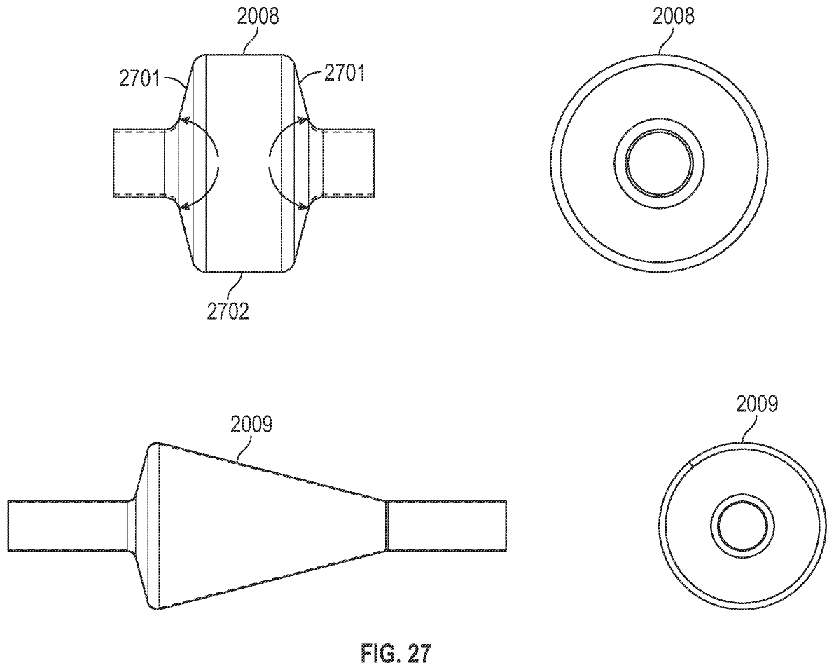

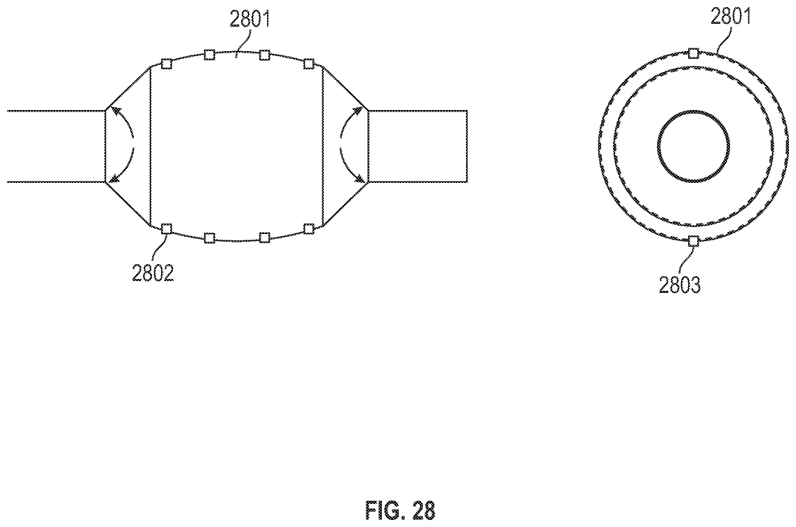

FIG. 26 shows a non-limiting example inlet check element 2008 in an inflated configuration according to some embodiments. In some embodiments, one or more outer portions 2601 of the check element 2008 comprise a higher density than one or more inner portions 2602. In some embodiments, while exact dimensions may vary, empirical data has shown that a minimum wall thickness of approximately 0.0007″ is acceptable to maintain structural integrity in one or more elements. FIG. 27 shows a non-limiting example side and font view for a directional pumping element 2001 according to some embodiments. FIG. 28 shows a non-limiting example inflated bi-directional pumping element 2801 according to some embodiments. In some embodiments, one or more bi-directional pumping elements 2801 comprises one or more magnetic and/or magnet material 2802 configured to aid in shaping the bi-directional pumping element inflation and/or deflation as previously described. In some embodiments, one or more bi-directional pumping elements 2801 comprises one or more fluid ports 2803 configured to apply vacuum and/or pressure to aid in shaping the bi-directional pumping element inflation and/or deflation as previously described.

In some embodiments, the system includes one or more balloons comprising varying wall thickness and/or densities. In some embodiments, the system includes a separate inflatable structure between the gas and balloon to perform this function. In some embodiments, one or more balloons comprises one or more ribs including one or more rib configurations. In some embodiments, the one or more ribs are configured to create varying resistance across the one or more balloons. In some embodiments, the varying resistance is configured to force a desired inflation direction. In some embodiments, the varying resistance is a result of the varying density in balloon wall thickness.

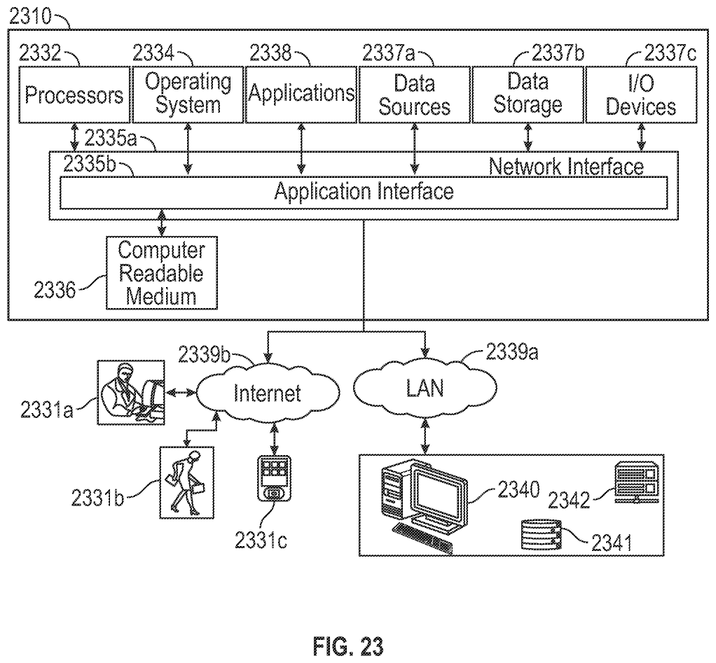

FIG. 23 illustrates a computer system 2310 enabling or comprising the systems and methods in accordance with some embodiments. In some embodiments, the computer system 2310 is configured to operate and/or process computer-executable code of one or more software modules of the aforementioned system and method. Further, in some embodiments, the computer system 2310 is configured to operate and/or display information within one or more graphical user interfaces (e.g., HMIs) integrated with or coupled to the system.

In some embodiments, the computer system 2310 comprises one or more processors 2332. In some embodiments, at least one processor 2332 resides in, or is coupled to, one or more servers. In some embodiments, the computer system 2310 includes a network interface 2335a and an application interface 2335b coupled to the least one processor 2332 capable of processing at least one operating system 2334. Further, in some embodiments, the interfaces 2335a, 2335b coupled to at least one processor 2332 are configured to process one or more of the software modules (e.g., such as enterprise applications 2338). In some embodiments, the software application modules 2338 includes server-based software. In some embodiments, the software application modules 2338 are configured to host at least one user account and/or at least one client account, and/or configured to operate to transfer data between one or more of these accounts using one or more processors 2332.

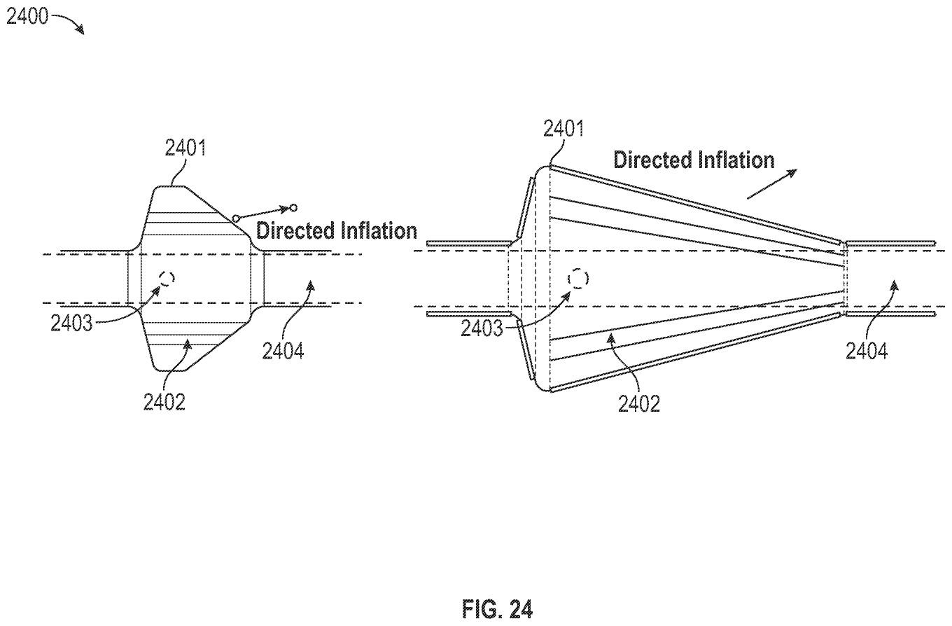

In some embodiments, the system comprises one or more pleated check and/or pumping elements. FIG. 24 shows a pleated element arrangement 2400 in a partially inflated configuration according to some embodiments. In some embodiments, one or more pleated elements 2401 include one or more pleated balloons 2401. In some embodiments, one or more pleats 2402 are configured to cause a pleated element to lay down on a “filler mandrel” in an even and/or organized way 2501. This allows for a smaller diameter balloon when deflated according to some embodiments. In some embodiments, the pleated element comprises one or more fill ports 2403 which is configured to receive element fluid from a mandrel 2404 as previously described.

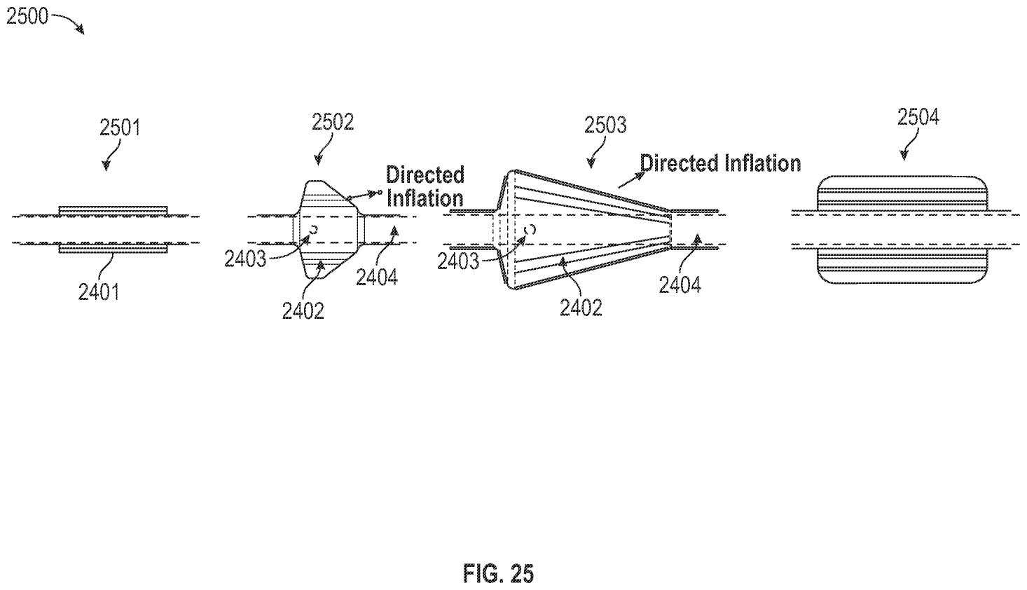

FIG. 25 illustrates the directional inflation 2500 of a pleated element according to some embodiments. In some embodiments, in a deflated state, the pleated element 2401 is configured to lay flat against the mandrel 2404. In some embodiments, upon receiving an initial element fluid through fill port 2403 the pleated element is configured to inflate to the shape 2502 which forces liquid forward. In some embodiments, as element fluid continues to pressurize the pleated element 2401 the pleated element 2401 continues to expand in the inflation direction 2503. In some embodiments, once a maximum pressure is achieved the pleated element is fully expanded inside a tube or stent as previously described.

With the above embodiments in mind, it is understood that the system is configured to implements various computer-implemented program steps involving data stored one or more non-transitory computer media according to some embodiments. In some embodiments, the above-described databases and models described throughout this disclosure are configured to store analytical models and other data on non-transitory computer-readable storage media within the computer system 2310 and on computer-readable storage media coupled to the computer system 2310 according to some embodiments. In addition, in some embodiments, the above-described applications of the system are stored on computer-readable storage media within the computer system 2310 and on computer-readable storage media coupled to the computer system 2310. In some embodiments, these operations are those requiring physical manipulation of structures including electrons, electrical charges, transistors, amplifiers, receivers, transmitters, and/or any conventional computer hardware in order to transform an electrical input into a different output. In some embodiments, these structures include one or more of electrical, electromagnetic, magnetic, optical, and/or magneto-optical signals capable of being stored, transferred, combined, compared, and otherwise manipulated. In some embodiments, the computer system 2310 comprises at least one computer readable medium 2336 coupled to at least one of at least one data source 2337a, at least one data storage 2337b, and/or at least one input/output 2337c. In some embodiments, the computer system 2310 is embodied as computer readable code on a computer readable medium 2336. In some embodiments, the computer readable medium 2336 includes any data storage that stores data, which is configured to thereafter be read by a computer (such as computer 2340). In some embodiments, the non-transitory computer readable medium 2336 includes any physical or material medium that is used to tangibly store the desired information, steps, and/or instructions and which is configured to be accessed by a computer 2340 or processor 2332. In some embodiments, the non-transitory computer readable medium 2336 includes hard drives, network attached storage (NAS), read-only memory, random-access memory, FLASH based memory, CD-ROMs, CD-Rs, CD-RWs, DVDs, magnetic tapes, and/or other optical and non-optical data storage. In some embodiments, various other forms of computer-readable media 2336 are configured to transmit or carry instructions to one or more remote computers 2340 and/or at least one user 2331, including a router, private or public network, or other transmission or channel, both wired and wireless. In some embodiments, the software application modules 2338 are configured to send and receive data from a database (e.g., from a computer readable medium 2336 including data sources 2337a and data storage 2337b that comprises a database), and data is configured to be received by the software application modules 2338 from at least one other source. In some embodiments, at least one of the software application modules 2338 are configured to be implemented by the computer system 2310 to output data to at least one user 2331 via at least one graphical user interface rendered on at least one digital display.

In some embodiments, the one or more non-transitory computer readable 2336 media are distributed over a conventional computer network via the network interface 2335a where some embodiments stored the non-transitory computer readable media are stored and executed in a distributed fashion. For example, in some embodiments, one or more components of the computer system 2310 are configured to send and/or receive data through a local area network (“LAN”) 2339a and/or an internet coupled network 2339b (e.g., such as a wireless internet). In some embodiments, the networks 2339a, 2339b include one or more wide area networks (“WAN”), direct connections (e.g., through a universal serial bus port), or other forms of computer-readable media 2336, and/or any combination thereof.

In some embodiments, components of the networks 2339a, 2339b include any number of personal computers 2340 which include for example desktop computers, laptop computers, and/or any fixed, generally non-mobile internet appliances coupled through the LAN 2339a. For example, some embodiments include one or more personal computers 2340, databases 2341, and/or servers 2342 coupled through the LAN 2339a that are configured for use by any type of user including an administrator. Some embodiments include one or more personal computers 2340 coupled through network 2339b. In some embodiments, one or more components of the computer system 2310 are configured to send or receive data through an internet network (e.g., such as network 2339b). For example, some embodiments include at least one user 2331a, 2331b, coupled wirelessly and accessing one or more software modules of the system including at least one enterprise application 2338 via an input and output (“I/O”) 2337c. In some embodiments, the computer system 2310 is configured to enable at least one user 2331a, 2331b, to be coupled to access enterprise applications 2338 via an I/O 2337c through LAN 2339a. In some embodiments, the user 2331 includes a user 2331a coupled to the computer system 2310 using a desktop computer, and/or laptop computers, or any fixed, generally non-mobile internet appliances coupled through the internet 2339b. In some embodiments, the user includes a mobile user 2331b coupled to the computer system 2310. In some embodiments, the user 2331b connects using any mobile computing 2331c to wireless coupled to the computer system 2310, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of heart assist pumps by actuating one or more inflatable elements to move fluid. The disclosure describes the specifics of how a machine including one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. Any figure depicting a content for display on a graphical user interface is a disclosure of the system configured to generate the graphical user interface and configured to display the contents of the graphical user interface. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. All flowcharts presented herein represent computer implemented steps and/or are visual representations of algorithms implemented by the system. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for pumping blood comprising:
one or more element fluid pumps,
one or more element tubes,
one or more elements, and
a stent,
wherein the one or more elements are housed within the stent;
wherein the one or more elements are configured to receive a fluid from the one or more element tubes; and
wherein the one or more element pumps are configured to execute an inflation and/or a deflation of the one or more elements via the one or more element tubes;

2. The system of claim 1,
further including one or more element housings;
wherein the one or more elements are housed in the one or more element housings;
wherein the one or more element housings are housed within the stent;
wherein the one or more elements are configured to pump a liquid though the one or more element housings as a result of the inflation and/or the deflation of the one or more elements.

3. The system of claim 1,
wherein the one or more elements are configured to execute a directional inflation; and
wherein the direction inflation is configured to force a liquid in one direction.

4. The system of claim 1,
wherein each of the one or more elements comprise an element inlet end and an outlet end;
wherein each the one or more elements are configured to enable an element inlet end to expand before an outlet end expands.

5. The system of claim 1,
wherein at least one of the one or more elements include a tubular shape;
wherein the tubular shape includes an element inlet end, an element outlet end, and an element hollow center portion; and
wherein the tubular shape is configured to enable a liquid to flow through the element hollow center portion.

6. The system of claim 5,
wherein the at least one element is configured to form the element hollow center portion when the at least one element is deflated.

7. The system of claim 2,
further including a mandrel;
wherein the mandrel is positioned within a center portion of the one or more element housings in the stent;
wherein the one or more elements are coupled to the mandrel;
wherein the one or more elements are configured to execute an inflation to cause one or more elements to expand from the mandrel toward the one or more element housings; and
wherein the expansion is configured to cause liquid to be pumped out of the housing in a single direction.

8. The system of claim 2,
wherein the one or more elements include a first element and a second element.

9. The system of claim 8,
wherein the first element comprises a first inflated volume;
wherein the second element comprises a second inflated volume; and
wherein the first inflated volume is less than the second inflated volume.

10. The system of claim 9,
wherein the first element is positioned before the second element in the one or more housings relative to pumping direction;
wherein inflation of the first element is configured to close an housing inlet end into the one or more housings; and
wherein inflation of the second element is configured to pump liquid from a first outlet end of the first element to a second outlet end of the second element.

11. The system of claim 10,
further comprising a third element;
wherein the third element comprises a third inflated volume;
wherein the third inflated volume is less than the second inflated volume.

12. The system of claim 11,
wherein the third element is positioned after the second element in the one or more housings relative to pumping direction; and
wherein inflation of the third element is configured to close a housing outlet end out the one or more housings.

13. The system of claim 2, further comprising:
a controller,
a first element, and
a second element;
wherein the one or more elements include the first element, and the second element; and
wherein the controller is configured to execute a first inflation of the first element before executing a second inflation of the second element.

14. The system of claim 13, further comprising:
a third element;
wherein the one or more elements include the third element; and
wherein the controller is configured to execute a third inflation of the third element after executing the second inflation of the second element.

15. The system of claim 2, further comprising:
a graphical user interface (GUI);
wherein the GUI is configured to enable a user to execute a first pumping sequence configured to pump liquid in a first direction; and
wherein the GUI is configured to enable a user to execute a second pumping sequence configured to pump liquid in a second direction.
